(19) United States
(12) Patent Application Publication
(10) Pub. No.: US 2024/0010679 A1
(43) Pub. Date: Jan. 11, 2024

(54) ANTINOCICEPTIVE COMPOUNDS AND USES THEREOF

(71) Applicant: THE UNIVERSITY OF BRITISH COLUMBIA, Vancouver (CA)

(72) Inventors: Shyh-Dar LI, Vancouver (CA); KK Durgra Rao VISWANADHAM, Vancouver (CA); Roland BOETTGER, Vancouver (CA)

(21) Appl. No.: 18/253,518

(22) PCT Filed: Nov. 18, 2021
(86) PCT No.: PCT/IB2021/060724

§ 371 Date: May 18, 2023

Related U.S. Application Data
(60) Provisional application No. 63/115,905, filed on Nov. 19, 2020.

(51) Int. Cl. C07K 7/06; A61P 25/24; A61P 25/04

(52) U.S. Cl. CPC C07K 7/06; A61P 25/24; A61P 25/04; A61K 38/00

(57) ABSTRACT

The present invention relates to anti-nociceptive compounds and uses thereof. In some embodiments, the present invention provides LEU-ENK analogues for treating pain, anxiety, mood disorders or depression.

### FIELD OF INVENTION

The present invention relates to anti-nociceptive compounds and uses thereof.

### BACKGROUND OF THE INVENTION

Opioids are often used for chronic pain treatment but may cause significant side effects. Exogenous opioids, such as morphine and fentanyl, are powerful analgesics for chronic pain¹. These drugs predominantly activate the mu opioid receptor (MOR), inducing multiple intracellular effects, such as modulating ion channels and second messengers that ultimately decrease neuronal activity²⁻³. MOR activation by these exogenous opioids also leads to side effects that limit their clinical use^{2,4-5}. Long-term use of these drugs may result in dependence and tolerance⁶. Respiratory depression may be the cause of death after overdose.

In addition to MOR, there are 2 major subtypes of opioid receptors (ORs) associated with analgesia⁷, including delta opioid receptor (DOR) and kappa opioid receptor (KOR). Although KOR agonists are potently analgesic and have been employed clinically in the treatment of pain, they produce side effects that limit their clinical use⁸. Difelikefalin and CR665 are D-amino-acid tetrapeptide agonists for KOR that are currently under clinical investigation as analgesics⁹⁻¹⁰. In general, activation of DOR has been shown to induce analgesia with fewer adverse effects^{2,11} on cardiovascular function¹²⁻¹⁴, gastrointestinal transit¹⁵, physical dependence¹⁶ and respiratory depression^{12-13,17} in preclinical models. In addition, DOR activation in murine studies has been linked to improvements in mood disorders, whereas genetic deletion of the DOR gene in mice appeared to increase anxiety².

Enkephalin (ENK) is a 5-amino acid peptide [sequence: YGGFL (Leu-ENK) or YGGFM (Met-ENK)], produced endogenously by neuronal and inflammatory cells for pain regulation¹⁸, that exhibits a 10 to 20-fold increased binding for DOR over MOR¹⁹. ENK is believed to be an effective and safer analgesic compared to morphine because it is generated endogenously and selective for DOR²⁰⁻²¹. ENK is however rapidly degraded and eliminated after administration⁵. Native Leu-ENK has a relatively short half-life (˜25 minutes in mouse plasma) and negligible antinociceptive activity in vivo. Accordingly, endogenous Leu-ENK, when introduced peripherally, exhibits poor stability and limited membrane permeability Mice deficient in the preproenkephalin gene exhibited increased levels of anxiety, suggesting a role for ENK in the management of emotional disorders as well as pain².

Several ENK analogues have been synthesized in attempts to improve potency, stability or brain penetration²²⁻³¹ or to reduce side effects caused by MOR agonists, such as respiratory depression, tolerance, physical dependence and constipation^{5,6,36-38,40,52-54} Some of these compounds displayed morphine-equivalent analgesic activity without inducing tolerance and dependence in animals²⁸ but have not been approved for human use after many years of development. The approaches utilized in generating the ENK analogues have included changing the peptide sequence, introducing unnatural amino acids, modifying the side chain, or attaching a carbohydrate ligand that facilitates the blood brain barrier (BBB) crossing, resulting in alterations in receptor binding affinity, selectivity, and potency. In some cases, derivatization of the peptide sequence and side chain may result in switch from agonism to antagonism. In other attempts, the ENK sequence was preserved but the N-terminus was conjugated with a cinnamoyl group via a C6 to C16 lipidic linker (US patent application #US20100292158A1, ImmuPharma). A chitosan-based nanoparticle formulation was developed for intranasal delivery of native ENK to the brain⁵.

Chronic pain may involve peripheral tissue inflammation, which can lead to upregulation of ORs at the synapse and increased infiltration of inflammatory cells (lymphocytes, macrophages) that release ENK for enhanced agonist efficacy at peripheral nerve terminals³²⁻³⁴. However, membrane-bound enkephalinases are expressed next to ORs for rapid degradation of ENK, and the two major enzymes are neprilysin (NEP) and aminopeptidase N (APN)³⁵⁻³⁶. Accordingly, a different approach may be to inhibit ENK catabolism to increase ENK concentration at the site where pain is perceived^{6,20-21,37-38}. Compounds that inhibit both enzymes in the periphery, i.e. dual enkephalinase inhibitors (DENKIs), have been developed, including kelatorphan³⁹, RB compounds⁴⁰⁻⁴², PL compounds⁴³, opiorphin³⁷ and STR-324⁶.

These compounds induced OR-mediated analgesia in animal models without causing side effects such as dependence, tolerance, constipation and respiratory suppression³⁷⁻³⁸. However, both NEP and APN are responsible for catabolism of many other endogenous proteins and peptides and this dual inhibition may cause other side effects²⁰. The efficacy of DENKIs is expected to be greatly reduced in immunosuppressed patients (such as those with AIDS, cancer and diabetes) or extinguished in patients whose pain is not associated with inflammation²⁰⁻²¹, as in periphery ENK is released mainly from the inflammatory immune cells⁴⁴⁻⁴⁶.

### SUMMARY OF THE INVENTION

The present invention relates to anti-nociceptive compounds and uses thereof. In some embodiments, the present invention provides LEU-ENK analogues for treating pain, anxiety, mood disorders or depression.

In one aspect, the present invention provides a compound of Formula I:

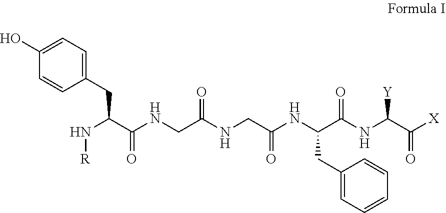

where R may be independently optionally substituted C₁₋₈ alkyl, C₁₋₈ alkenyl, acyl or phenyl;
Y may be CH₂CH(CH₃)₂ or CH₂CH₂SCH₃; and
X may be independently OH, NH₂, NH—NH₂, NH—NHOH, halo, thio, NCH₃, N(CH₃)₂, triazole, tetrazole, alkoxy or a methyl ester, or a pharmaceutically acceptable salt thereof.

In some embodiments, R may be optionally substituted with halo or thio.

In some embodiments, R may be acyl or C₁₋₈ alkyl.

In some embodiments, R may be alkanoyl or benzoyl.

In some embodiments, R may be pivaloyl or t-butyl.

In some embodiments, R may be 2-(tert-butyl)benzoyl, 2,4,6-tri-tert-butylbenzoyl, 2,2-diphenylpropanal, 2-hydroxy-2,2-diphenylacetyl, 3,3-dimethyl-2-phenylbutanoyl, 2-methyl-2-phenylpropanoyl, 2-hydroxy-2-phenylpropanoyl, 2-methyl-2-phenylpropanoyl, 2-amino-2,2-diphenylacetyl, dihydroxyl phenyl acetyl, trihydroxyl phenyl acetyl, dimethoxy phenylacetyl or trimethoxy phenylacetyl.

In some embodiments, R may be (CF₃)₂—CHOH—CO, HO—CHCH₂OH—CO, (CH₃)₂—COH—CO, NH₂—CH—(CH₃)₂—CO, (CH₃)₃—CH₂CO, or (CH₃)₂—CHOH—CH₂—.

In some embodiments, R may be

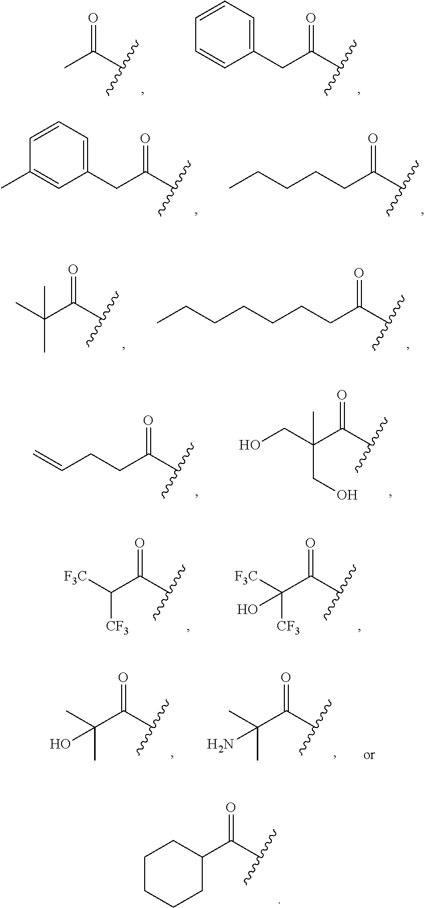

In some embodiments, R may be

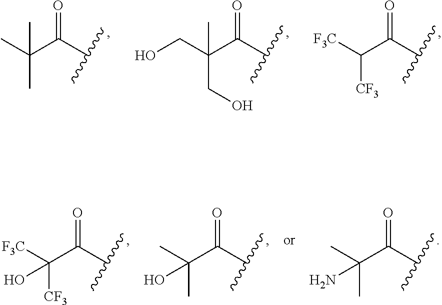

In some embodiments, R may be

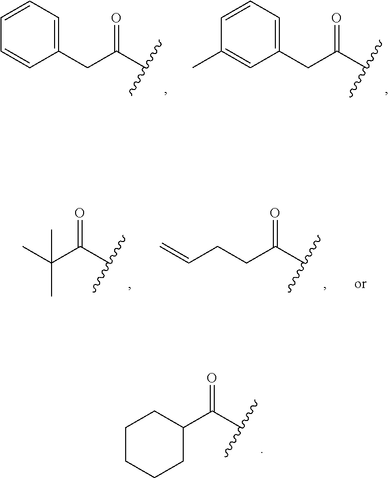

In some embodiments, R may be

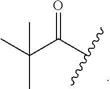 [SMILES: *C(=O)C(C)(C)C]

In some embodiments, X may be independently OH or methyl ester.

In some embodiments,
R may be

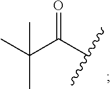 [SMILES: *C(=O)C(C)(C)C]

Y may be CH₂CH(CH₃)₂; and
X may be independently OH or a methyl ester, or a pharmaceutically acceptable salt thereof.

In some embodiments, the compound may have the following structure:

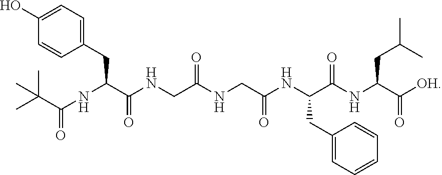 [SMILES: CC(C)C[C@H](NC(=O)[C@H](Cc1ccccc1)NC(=O)CNC(=O)CNC(=O)[C@H](Cc1ccc(O)cc1)NC(=O)C(C)(C)C)C(=O)O]

In alternative aspects, the invention may provide a pharmaceutical composition including a compound as described herein.

In alternative aspects, the invention provides a method of treating pain, anxiety, a mood disorder or depression, by administering a compound or a pharmaceutical composition as described herein, to a subject in need thereof.

In alternative aspects, the invention provides the use of a compound or a pharmaceutical composition as described herein, for treating pain, anxiety, a mood disorder or depression in a subject in need thereof.

In alternative aspects, the invention provides a commercial package including of a compound or a pharmaceutical composition as described herein, together with instructions for use in treating pain, anxiety, a mood disorder or depression.

In some embodiments, the pain may be acute pain, chronic pain, inflammatory pain, or neuropathic pain.

In some embodiments, the subject may be a human.

This summary of the invention does not necessarily describe all features of the invention.

### BRIEF DESCRIPTION OF THE DRAWINGS

These and other features of the invention will become more apparent from the following description in which reference is made to the appended drawings wherein:

FIG. 1 shows the chemical structure of Leu-ENK with C-terminal and N-terminal modifications where X denotes the C-terminus, which was either unmodified (Carboxylic acid, OH) or contained a Methyl ester (OMe), the arrows (labelled 1 and 2) indicate the major cleavage sites and R denotes a set of eight hydrophobic modifications introduced at the N-terminus as shown in Table 1;

FIG. 2 shows a general synthesis scheme for preparing Leu-ENK analogues where (A) Di-tert-butyl decarbonate (Boc anhydride), NaOH, dioxane/H₂O, 4 h; (B) Methyl L-phenylalanine, N,N′-diisopropylethylamine (DIPEA), 1-Ethyl-3-(3-dimethylaminopropyl) carbodiimide hydrochloride (EDC-HCl), 4-dimethylaminopyridine (4-DMAP), tetrahydrofuran (THF), 1 h at 0° C.; 12 h at 23° C.; (C) LiOH, THF, 1 h, room temperature (rt); (D) Methyl L-leucine, DIPEA, EDC-HCl, 4-DMAP, THF, 1 h at 0° C.; 12 h at 23° C.; (E) Trifluoroacetic acid (TFA), dichloromethane (DCM), 1 h, rt. (Fa) Acyl chloride (RCOCl), L-tyrosine, DIPEA, EDC-HCl, 4-DMAP, THF, 1 h at 0° C. at 12 h at 23° C. (Fb) N-acyl-L-tyrosine, N,N′-dicyclohexylcarbodiimide (DCC), hydroxybenzotriazole (HOBt), dimethylformamide (DMF), DIPEA, 1 h at 0° C.; 7 h at 23° C.;

FIG. 3A is a graph showing KK-102 (triangles) in mouse plasma rapidly deesterifies to KK-103 (squares);

FIG. 3B is a graph showing KK-103 is stable in mouse plasma for at least 5 h with an extrapolated half-life of 36 h. Data=mean±SD, n=3.

FIG. 4 shows the chemical structure of KK-102 and KK-103 containing C-terminal Methyl ester or carboxylic acid, respectively, while both peptides contain an N-terminal pivaloyl group;

FIG. 5A is a graph showing the stability of KK-103 (squares) in human cerebrospinal fluid compared to its parent Leu-ENK (triangles). Data=mean±SD, n=3. Statistical significance with ***: P<0.001;

FIG. 5B is a graph showing the stability of KK-103 (squares) in mouse plasma compared to its parent drug Leu-ENK (triangles). Data=mean±SD, n=3.

Statistical significance with ***: P<0.001;

FIG. 6 is a graph showing antinociceptive activity of KK-102 (triangles) and KK-103 (squares) compared to Leu-ENK (diamonds) in female CD1 mice using a hot plate test after s.c. injection at 20 μmol/kg. Data=mean±SEM, n=6;

FIG. 7 is a graph showing antinociceptive activity of KK-103 (squares) (13 mg/kg) and morphine (circles) (10 mg/kg) in female CD1 mice determined by the hot plate test after s.c. injection. Data=mean±SEM, n=6. Statistical significance with *: P<0.05; **: P<0.002; ***: P<0.001;

FIG. 8 is a graph showing antinociceptive activity of KK-103 (13 mg/kg, s.c.) (squares) in female CD1 mice determined by the hot plate test when co-delivered with naloxone (circles) or methylnaloxone (triangles). Data=mean±SEM, n=6. Statistical significance analysis comparing KK-103 vs. KK-103+naloxone: **: P<0.002 ***: P<0.001;

FIG. 9 is a graph showing antinociceptive activity of Leu-ENK (circles), KK-103 (squares) and vehicle (triangles) in female CD1 mice determined by the hot-plate test after intranasal administration at 2 μmol/kg. Data=mean±SEM, n=4-6;

FIG. 10A is a graph showing dependence effect of KK-103 and morphine in female CD1 mice. Mice received twice daily treatments of KK-103 (13 mg/kg/dose) or morphine (10 mg/kg/dose) for 7 days. Two hours post final-dose, mice received 4 mg/kg naloxone and the #of jumps within 10 min were measured. Data=mean±SEM, n=6. Statistical significance with *: P<0.05;

FIG. 10B is a graph showing change of analgesic activity of KK-103 and morphine following long-term treatments. Mice received twice-daily treatments of KK-103 (13 mg/kg/dose) or morphine (10 mg/kg/dose) for 7 days. The analgesic activity was measured on day 1, 4, 7 using hot plate test and expressed as AUC₀₋₅ h of the antinociceptive plot. *=p<0.05;

FIG. 10C is a graph showing breathing rates of female CD1 mice at different time points after s.c. injection of KK-103 (13 mg/kg) or morphine (10 mg/kg). Data were analyzed in a blinded manner and expressed as mean+95% CI, n=6. Statistical significance with *=p<0.05;

FIG. 11 shows the results of a formalin injection test comparing KK-103 (13 mg/kg, (squares), morphine (10 mg/kg, diamonds) and vehicle (triangles) with responses representing the total time of mice spent licking and biting in phase 1 (0-5 min) and phase 2 (20-40 min) of the nociceptive response to formalin injection. **P<0.001 compared to vehicle. Data=mean±95% confidence interval (n=10), Statistical comparisons were performed using a two-tailed unpaired t-test one-way ANOVA with Tukey post hoc test;

FIG. 12 shows the dose response relationship of KK-103 in a formalin injection test, where vehicle=closed triangles, 5 mg/kg=open triangles, 10 mg/kg=open inverted triangles, 20 mg/kg=diamonds, and 50 mg/kg=squares. Data points represent the total licking and biting time of mice in phase 1 (0-5 min) and phase 2 (20-40 min) as a nociceptive response to formalin injection. **P<0.008 compared to vehicle. Data=mean±95% confidence interval (n=8), Statistical comparisons were performed using a one-way ANOVA with Tukey post hoc test;

FIG. 13 shows the effect of KK-103 (squares) and morphine (diamonds) on respiratory rate after s.c. drug administration (morphine 10 mg/kg, KK-103 13 mg/kg). **P<0.0001, *P<0.05 compared to vehicle (triangles). Data=mean±95% confidence interval (n=6). Statistical comparisons using one-way ANOVA with Tukey post hoc test (A), one-way ANOVA with Turkey post hoc test (B), or a two-tailed unpaired t-test (C);

FIG. 14 shows the plasma concentration of KK-103 in female CD-1 mice at a dose of 50 mg/kg. KK-103 was injected s.c. (triangles) or i.v. (squares). Data is plotted as mean±SD (n=6);

FIG. 15 shows the results of a Forced Swim Test of mice treated with vehicle, KK-103, or desipramine. Data points represent the immobility score of mice after treatment with vehicle, KK-103 (15 mg/kg s.c.) or desipramine (30 mg/kg i.p.). Data=mean±SD (n=18), Statistical comparisons were performed using a one-way ANOVA with Tukey post hoc test. *p<0.05; ***p<0.005;

FIG. 16 shows the results of a Tail suspension test of mice treated with vehicle, KK-103, or desipramine. Data points represent the immobility score of mice after treatment with vehicle, KK-103 (15 mg/kg s.c.) or desipramine (30 mg/kg i.p.). Data=mean±SD (n=7-8), Statistical comparisons were performed using a one-way ANOVA with Tukey post hoc test. *p<0.05; **p<0.01; and

FIG. 17 shows the effects of vehicle, KK-103, or desipramine on locomotion determined in the open field test. Data=mean±95% confidence interval (n=10), Statistical comparisons were performed using one-way ANOVA with Tukey post hoc test. **p<0.05.

### DETAILED DESCRIPTION

The present disclosure provides, in part, compounds, such as Leu-ENK analogues, with improved antinociceptive activity over native or endogenous ENK peptide, such as Leu-ENK (YGGFL) or Met-ENK (YGGFM). In some embodiments, the present disclosure provides compounds, such as Leu-ENK analogues, with enhanced metabolic stability over native ENK. In some embodiments, the present disclosure provides compounds, such as Leu-ENK analogues, that are capable of crossing the BBB.

The present disclosure provides, in part, a compound of Formula I:

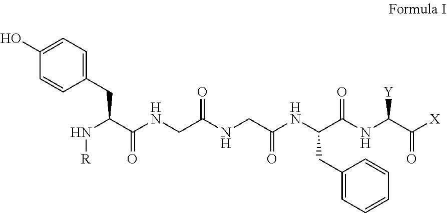

where R may be independently optionally substituted C₁₋₈ alkyl, C₁₋₈ alkenyl, acyl or phenyl;
Y may be CH₂CH(CH₃)₂ or CH₂CH₂SCH₃; and
X may be independently OH, NH₂, NH—NH₂, NH—NHOH, halo, thio, NCH₃, N(CH₃)₂, triazole, tetrazole, or alkoxy.

In some embodiments R may be optionally substituted with halo or thio.

In some embodiments R may be acyl or alkyl.

In some embodiments R may be alkanoyl or benzoyl.

In some embodiments R may be pivaloyl or t-butyl.

In some embodiments R may be 2-(tert-butyl)benzoyl, 2,4,6-tri-tert-butylbenzoyl, 2,2-diphenylpropanal, 2-hydroxy-2,2-diphenylacetyl, 3,3-dimethyl-2-phenylbutanoyl, 2-methyl-2-phenylpropanoyl, 2-hydroxy-2-phenylpropanoyl, 2-methyl-2-phenylpropanoyl, 2-amino-2,2-diphenylacetyl, dihydroxyl phenyl acetyl, trihydroxyl phenyl acetyl, dimethoxy phenylacetyl or trimethoxy phenylacetyl.

In some embodiments R may be (CF₃)₂—CHOH—CO, HO—CHCH₂OH—CO, (CH₃)₂—COH—CO, NH₂—CH—(CH₃)₂—CO, (CH₃)₃—CH₂CO, or (CH₃)₂—CHOH—CH₂—.

In some embodiments X may be independently OH or methyl ester.

By “acyl” is meant a group having the structure C(O)R¹, where R¹ may be alkyl, alkenyl, alkynyl, cycloalkyl, cycloalkylalkyl, aryl, arylalkyl or alkylarylalkyl. Unless stated otherwise specifically herein, the term “acyl” is meant to include acyl groups optionally substituted by one or more substituents as described herein. Accordingly, in some embodiments, R¹ may be branched alkyl optionally substituted with one or more of halo (e.g., fluoro), nitro, methoxy, hydroxyl, amino, or thio. In alternative embodiments, R¹ may be branched alkyl optionally substituted with one or more of halo (e.g., fluoro), hydroxyl, or amino.

“Alkyl” refers to a straight, branched or cyclic (cycloalkyl) hydrocarbon chain group consisting solely of carbon and hydrogen atoms, containing no unsaturation and including, for example, from one to ten carbon atoms, such as 1, 2, 3, 4, 5, 6, 7, 8, 9, or 10 carbon atoms, and which is attached to the rest of the molecule by a single bond. In alternative embodiments, the alkyl group may contain from one to eight carbon atoms, such as 1, 2, 3, 4, 5, 6, 7, or 8 carbon atoms. In alternative embodiments, the alkyl group may contain from one to six carbon atoms, such as 1, 2, 3, 4, 5, or 6 carbon atoms. In alternative embodiments, the alkyl group may contain from two to ten carbon atoms, such as 2, 3, 4, 5, 6, 7, 8, 9, or 10 carbon atoms. In alternative embodiments, the alkyl group may contain from two to eight carbon atoms, such as 2, 3, 4, 5, 6, 7, or 8 carbon atoms. In alternative embodiments, the alkyl group may contain from two to six carbon atoms, such as 2, 3, 4, 5, or 6 carbon atoms. In alternative embodiments, the alkyl group may be branched. In alternative embodiments, straight hydrocarbon chains may be specifically excluded. Unless stated otherwise specifically in the specification, the alkyl group may be optionally substituted by one or more substituents as described herein. Unless stated otherwise specifically herein, it is understood that the substitution can occur on any carbon of the alkyl group.

“Alkenyl” refers to a straight or branched hydrocarbon chain group consisting solely of carbon and hydrogen atoms, containing at least one double bond and including, for example, from two to ten carbon atoms, such as 2, 3, 4, 5, 6, 7, 8, 9, or 10 carbon atoms, and which is attached to the rest of the molecule by a single bond or a double bond. In alternative embodiments, the alkenyl group may contain from two to eight carbon atoms, such as 2, 3, 4, 5, 6, 7, or 8 carbon atoms. In alternative embodiments, the alkenyl group may contain from three to six carbon atoms, such as 3, 4, 5, or 6 carbon atoms. In alternative embodiments, the alkyl group may be branched. In alternative embodiments, straight hydrocarbon chains may be specifically excluded. Unless stated otherwise specifically in the specification, the alkenyl group may be optionally substituted by one or more substituents as described herein. Unless stated otherwise specifically herein, it is understood that the substitution can occur on any carbon of the alkenyl group.

“Alkynyl” refers to a straight or branched hydrocarbon chain group consisting solely of carbon and hydrogen atoms, containing at least one triple bond and including, for example, from two to ten carbon atoms. In alternative embodiments, the alkynyl group may contain from two to eight carbon atoms, such as 2, 3, 4, 5, 6, 7, or 8 carbon atoms. In alternative embodiments, the alkynyl group may contain from three to six carbon atoms, such as 3, 4, 5, or 6 carbon atoms. In alternative embodiments, the alkyl group may be branched. In alternative embodiments, straight hydrocarbon chains may be specifically excluded. Unless stated otherwise specifically in the specification, the alkynyl group may be optionally substituted by one or more substituents as described herein.

“Cycloalkyl” refers to a stable monovalent monocyclic, bicyclic or tricyclic hydrocarbon group consisting solely of carbon and hydrogen atoms, having for example from 3 to 15 carbon atoms, and which is saturated and attached to the rest of the molecule by a single bond. In alternative embodiments, the cycloalkyl group may contain from three to six carbon atoms, such as 3, 4, 5, or 6 carbon atoms. Unless otherwise stated specifically herein, the term “cycloalkyl” is meant to include cycloalkyl groups which are optionally substituted as described herein.

“Cycloalkylalkyl” refers to a group of the formula —RₐR_{d}, where Rₐ is an alkyl group as described herein and R_{d} is a cycloalkyl group as described herein. The cycloalkylalkyl group(s) may be optionally substituted as described herein.

“Aryl” refers to a mono- or bicyclic aromatic ring containing only carbon atoms, including for example, 6-14 members, such as 6, 7, 8, 9, 10, 11, 12, 13, or 14 members. Examples of aryl groups include phenyl, biphenyl, naphthyl, indanyl, indenyl, etc. Unless stated otherwise specifically herein, the term “aryl” is meant to include aryl groups optionally substituted by one or more substituents as described herein.

“Arylalkyl” refers to a group of the formula —RₐR_{b} where Rₐ is an alkyl group as described herein and R_{b} is one or more aryl moieties as described herein. The arylalkyl group(s) may be optionally substituted as described herein.

“Alkoxy” refers to a group of the formula —OR_{c}, where R_{c} is a C₁₋₁₀ alkyl or a C₁₋₈ alkyl group or a C₁₋₆ alkyl group as described herein. The alkyl group(s) may be optionally substituted as described herein. Alkoxy groups include without limitation —OCH₃, —OCH₂CH₃, propyloxy, t-butyloxy, pivaloyloxy, ethenoxy, propenoxy, cyclhexonoxy, or substituted benzyloxy groups.

Substitutions may include, without limitation, halo, nitro, methoxy, hydroxyl, amino, thio, etc. substitutions.

“Halo” refers to bromo, chloro, fluoro, iodo, etc. In some embodiments, suitable halogens include fluorine or chlorine.

“Optional” or “optionally” means that the subsequently described event of circumstances may or may not occur, and that the description includes instances where said event or circumstance occurs one or more times and instances in which it does not. For example, “optionally substituted alkyl” means that the alkyl group may or may not be substituted and that the description includes both substituted alkyl groups and alkyl groups having no substitution, and that said alkyl groups may be substituted one or more times. Examples of optionally substituted alkyl groups include, without limitation, methyl, ethyl, propyl, etc. and including cycloalkyls such as cyclopropyl, cyclobutyl, cyclopentyl, cyclohexyl, cycloheptyl, etc.; examples of optionally substituted alkenyl groups include allyl, crotyl, 2-pentenyl, 3-hexenyl, 2-cyclopentenyl, 2-cyclohexenyl, 2-cyclopentenylmethyl, 2-cyclohexenylmethyl, etc. In some embodiments, optionally substituted alkyl and alkenyl groups include C₁₋₆ alkyls or alkenyls. In some embodiments, optionally substituted benzyloxy groups include halo (e.g., fluoro, bromo, chloro), nitro, methoxy, hydroxy, and/or amino substitutions.

In some embodiments R may be

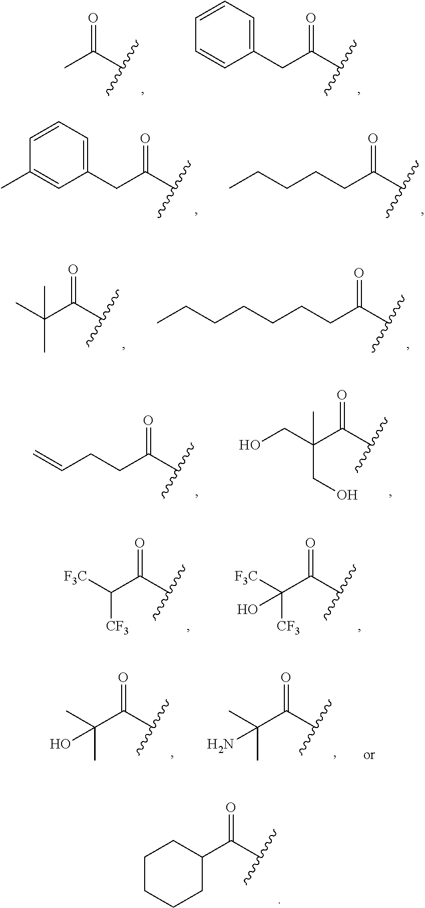

In some embodiments R may be

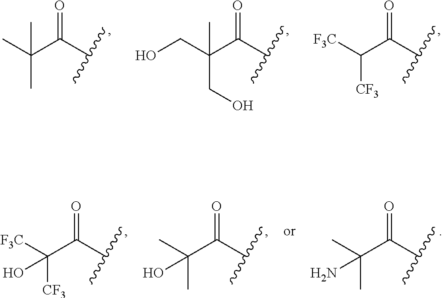

In some embodiments R may be

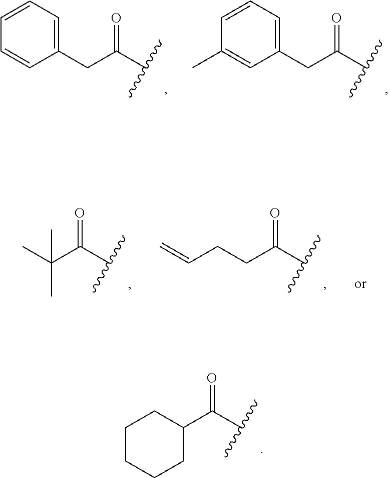

In some embodiments R may be

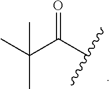 [SMILES: *C(=O)C(C)(C)C]

In some embodiments, a compound according to the present disclosure may have one or more of the following structures:

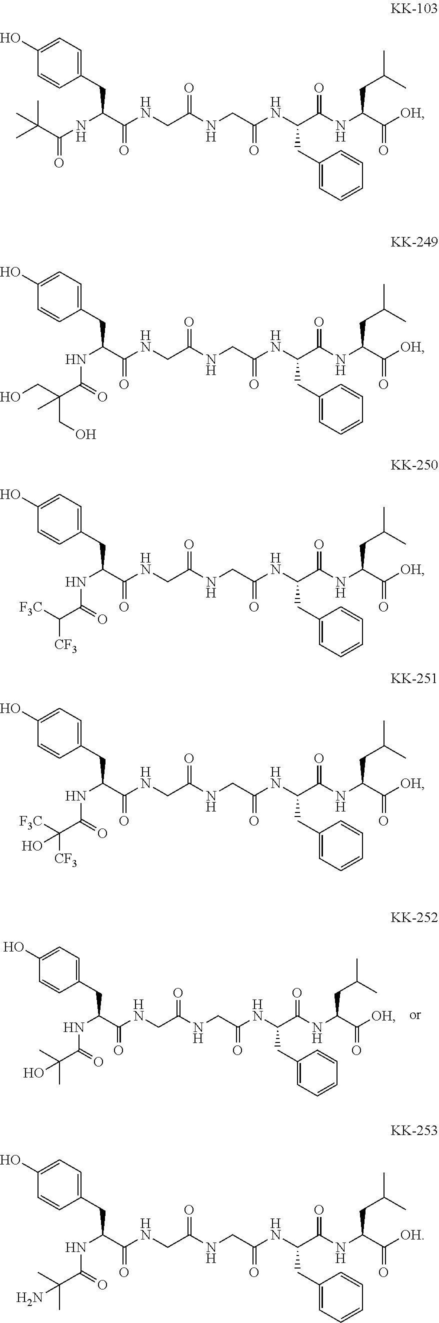

In some embodiments a compound according to the present disclosure may have the following structure:

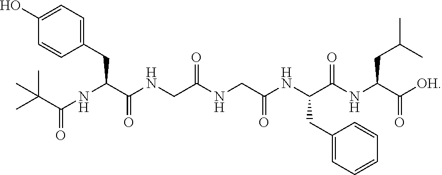 [SMILES: CC(C)C[C@H](NC(=O)[C@H](Cc1ccccc1)NC(=O)CNC(=O)CNC(=O)[C@H](Cc1ccc(O)cc1)NC(=O)C(C)(C)C)C(=O)O]

In some embodiments, a compound according to the present disclosure may be supplied as a “prodrug” or as a protected form, which releases the compound after administration to a subject. For example, a compound may carry a protective group which is split off by hydrolysis in body fluids, e.g., in the bloodstream, thus releasing the active compound or is oxidized or reduced in body fluids to release the compound. Accordingly, a “prodrug” is meant to indicate a compound that may be converted under physiological conditions or by solvolysis to a biologically active compound of the invention. Thus, the term “prodrug” refers to a metabolic precursor of a compound of the invention that is pharmaceutically acceptable. A prodrug may be inactive when administered to a subject in need thereof and may be converted in vivo to an active compound of the invention. Prodrugs are typically rapidly transformed in vivo to yield the parent compound of the invention, for example, by hydrolysis in blood. The prodrug compound often offers advantages of solubility, tissue compatibility or delayed release in a subject.

The term “prodrug” is also meant to include any covalently bonded carriers which release the active compound of the invention in vivo when such prodrug is administered to a subject. Prodrugs of a compound of the invention may be prepared by modifying functional groups present in the compound of the invention in such a way that the modifications are cleaved, either in routine manipulation or in vivo, to the parent compound of the invention. Prodrugs include compounds of the invention where a hydroxy, amino or mercapto group is bonded to any group that, when the prodrug of the compound of the invention is administered to a mammalian subject, cleaves to form a free hydroxy, free amino or free mercapto group, respectively. Examples of prodrugs include, but are not limited to, acetate, formate and benzoate derivatives of alcohol and acetamide, formamide, and benzamide derivatives of amine functional groups in one or more of the compounds of the invention and the like.

A discussion of prodrugs may be found in “Smith and Williams' Introduction to the Principles of Drug Design,” H. J. Smith, Wright, Second Edition, London (1988); Bundgard, H., Design of Prodrugs (1985), pp. 7-9, 21-24 (Elsevier, Amsterdam); The Practice of Medicinal Chemistry, Camille G. Wermuth et al., Ch 31, (Academic Press, 1996); A Textbook of Drug Design and Development, P. Krogsgaard-Larson and H. Bundgaard, eds. Ch 5, pgs 113 191 (Harwood Academic Publishers, 1991); Higuchi, T., et al., “Pro-drugs as Novel Delivery Systems,” A.C.S. Symposium Series, Vol. 14; or in Bioreversible Carriers in Drug Design, ed. Edward B. Roche, American Pharmaceutical Association and Pergamon Press, 1987.

Suitable prodrug forms of one or more of the compounds of the disclosure may include embodiments in which one or more OH groups as set forth in Formula (I) may be protected as OC(O)RP, where RP may be optionally substituted C₁₋₆ alkyl. In these cases, the ester groups may be hydrolyzed in vivo (e.g. in bodily fluids), liberating the OH groups and releasing the active compounds. Prodrug embodiments of the invention may include compounds of Formula (I), where one or more OH groups may be protected with acetate, for example as OC(O)CH₃.

In some embodiments, formulations, preparation, and compositions including compounds according to the present disclosure can include mixtures of stereoisomers, individual stereoisomers, and enantiomeric mixtures, mixtures of multiple stereoisomers, double-bond isomers (i.e., geometric E/Z isomers) or diastereomers (e.g., enantiomers (i.e., (+) or (−)) or cis/trans isomers). In some embodiments, the chemical structures depicted herein, and therefore the compounds according to the present disclosure, encompass corresponding stereoisomers, that is, both the stereomerically pure form (e.g., geometrically pure, enantiomerically pure, or diastereomerically pure) and enantiomeric and stereoisomeric mixtures, e.g., racemates. In general, a compound may be supplied in any desired degree of chiral purity. In some embodiments, compounds according to the present disclosure may include a stereoisomer or a mixture of stereoisomers including the D and L configurations of an amino acid.

Enantiomeric and stereoisomeric mixtures of compounds according to the present disclosure can typically be resolved into their component enantiomers or stereoisomers by well-known methods, such as chiral-phase gas chromatography, chiral-phase high performance liquid chromatography, crystallizing the compound as a chiral salt complex, or crystallizing the compound in a chiral solvent. Enantiomers and stereoisomers can also be obtained from stereomerically or enantiomerically pure intermediates, reagents, and catalysts by well-known asymmetric synthetic methods.

As used herein the singular forms “a”, “and”, and “the” include plural referents unless the context clearly dictates otherwise. For example, “a compound” refers to one or more of such compounds, and equivalents thereof as known to those skilled in the art.

In some embodiments, one or more of the compounds disclosed herein, such as a compound according to Formula (I), may be provided in combination with a carrier, such as a pharmaceutically acceptable carrier.

In some embodiments, the present disclosure provides a pharmaceutical composition including a compound as disclosed herein, such as a compound according to Formula (I), or a pharmaceutically acceptable salt thereof and a pharmaceutically acceptable excipient. In some embodiments, pharmaceutical compositions including an effective amount of a compound as disclosed herein, such as a compound according to Formula (I), are provided. In some embodiments, the pharmaceutical composition may be for treating pain. By “pain” is meant, without limitation, acute, chronic or inflammatory pain, neuropathic pain etc. In some embodiments, the pharmaceutical composition may be for treating anxiety, mood disorders or depression.

A compound as disclosed herein, such as a compound according to Formula (I), and its pharmaceutically acceptable salts, enantiomers, solvates, or derivatives may be useful because it may have pharmacological activity in animals, including humans. In some embodiments, one or more of the compounds according to the disclosure may be stable in plasma, when administered to a subject, such as a human.

In general, a compound according to the disclosure may be administered to a subject in need thereof, or by contacting a cell or a sample, for example, with a pharmaceutical composition comprising a therapeutically effective amount of a compound as disclosed herein, such as a compound according to Formula (I).

In some embodiments, a compound as disclosed herein, such as a compound according to Formula (I), may be provided in combination with any other active agents or pharmaceutical compositions where such combined therapy may be useful to treat pain, anxiety, mood disorders or depression.

Combinations of compounds according to the disclosure, or for use according to the disclosure, and other agents useful for the treatment of pain, anxiety, mood disorders or depression may be administered separately or in conjunction. The administration of one agent may be prior to, concurrent to, or subsequent to the administration of other agent(s).

Compounds according to the disclosure, or for use according to the disclosure, may be provided alone or in combination with other compounds in the presence of a liposome, a nanoparticle, an adjuvant, or any pharmaceutically acceptable carrier, diluent or excipient, in a form suitable for administration to a subject. If desired, treatment with a compound according to the disclosure may be combined with more traditional and existing therapies for the therapeutic indications described herein. Compounds according to the disclosure may be provided chronically or intermittently. “Chronic” administration refers to administration of the compound(s) in a continuous mode as opposed to an acute mode, so as to maintain the initial therapeutic effect (activity) for an extended period of time. “Intermittent” administration is treatment that is not consecutively done without interruption, but rather is cyclic in nature. The terms “administration,” “administrable,” or “administering” as used herein should be understood to mean providing a compound of the disclosure to the subject in need of treatment.

“Pharmaceutically acceptable carrier, diluent or excipient” may include, without limitation, any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier that has been approved, for example, by the United States Food and Drug Administration or other governmental agency as being acceptable for use in humans or animals.

A compound of the present disclosure may be administered in the form of a pharmaceutically acceptable salt. In such cases, pharmaceutical compositions in accordance with this disclosure may comprise a salt of such a compound, preferably a physiologically acceptable salt, which are known in the art. In some embodiments, the term “pharmaceutically acceptable salt” as used herein means an active ingredient comprising compounds of Formula I, used in the form of a salt thereof, particularly where the salt form confers on the active ingredient improved pharmacokinetic properties as compared to the free form of the active ingredient or other previously disclosed salt form.

A “pharmaceutically acceptable salt” may include both acid and base addition salts. A “pharmaceutically acceptable acid addition salt” refers to those salts which retain the biological effectiveness and properties of the free bases, which are not biologically or otherwise undesirable, and which may be formed with inorganic acids such as hydrochloric acid, hydrobromic acid, sulfuric acid, nitric acid, phosphoric acid and the like, and organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

A “pharmaceutically acceptable base addition salt” refers to those salts which may retain the biological effectiveness and properties of the free acids, which may not be biologically or otherwise undesirable. These salts may be prepared from addition of an inorganic base or an organic base to the free acid. Salts derived from inorganic bases may include, but are not limited to, the sodium, potassium, lithium, ammonium, calcium, magnesium, iron, zinc, copper, manganese, aluminum salts and the like.

Preferred inorganic salts may be the ammonium, sodium, potassium, calcium, and magnesium salts. Salts derived from organic bases may include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins and the like. Particularly preferred organic bases may be isopropylamine, diethylamine, ethanolamine, trimethylamine, dicyclohexylamine, choline and caffeine.

Thus, the term “pharmaceutically acceptable salt” encompasses all acceptable salts including but not limited to acetate, lactobionate, benzenesulfonate, laurate, benzoate, malate, bicarbonate, maleate, bisulfate, mandelate, bitartarate, mesylate, borate, methylbromide, bromide, methylnitrite, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutame, stearate, glycollylarsanilate, sulfate, hexylresorcinate, subacetate, hydradamine, succinate, hydrobromide, tannate, hydrochloride, tartrate, hydroxynaphthoate, teoclate, iodide, tosylate, isothionate, triethiodide, lactate, panoate, valerate, and the like.

Pharmaceutically acceptable salts of a compound of the present disclosure may be used as a dosage for modifying solubility or hydrolysis characteristics, or may be used in sustained release or prodrug formulations. Also, pharmaceutically acceptable salts of a compound of this disclosure may include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide.

Pharmaceutical formulations may typically include one or more carriers acceptable for the mode of administration of the preparation. Suitable carriers may be those known in the art for use in such modes of administration.

Suitable pharmaceutical compositions may be formulated by means known in the art and their mode of administration and dose determined by the skilled practitioner. For parenteral administration, a compound may be dissolved in sterile water or saline or a pharmaceutically acceptable vehicle used for administration of non-water-soluble compounds such as those used for vitamin K. For enteral administration, the compound may be administered in a tablet, capsule or dissolved in liquid form. The table or capsule may be enteric coated, or in a formulation for sustained release. Many suitable formulations are known, including, polymeric or protein microparticles encapsulating a compound to be released, ointments, gels, hydrogels, or solutions which can be used topically or locally to administer a compound. A sustained release patch or implant may be employed to provide release over a prolonged period of time. Many techniques known to skilled practitioners are described in Remington: The Science & Practice of Pharmacy by Alfonso Gennaro, 20^{th} ed., Williams & Wilkins, (2000). Formulations for parenteral administration may, for example, contain excipients, polyalkylene glycols such as polyethylene glycol, oils of vegetable origin, or hydrogenated naphthalenes. Biocompatible, biodegradable lactide polymer, lactide/glycolide copolymer, or polyoxyethylene-polyoxypropylene copolymers may be used to control the release of a compound. Other potentially useful parenteral delivery systems for modulatory compounds may include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

A compound or a pharmaceutical composition according to the present disclosure may be administered by oral or non-oral, e.g., intramuscular, intraperitoneal, intravenous, intracisternal injection or infusion, subcutaneous injection, transdermal or transmucosal routes. In some embodiments, a compound or pharmaceutical composition in accordance with this disclosure may be administered by means of a medical device or appliance such as an implant, graft, prosthesis, stent, etc. Implants may be devised which are intended to contain and release such compounds or compositions. An example would be an implant made of a polymeric material adapted to release the compound over a period of time. A compound may be administered alone or as a mixture with a pharmaceutically acceptable carrier e.g., as solid formulations such as tablets, capsules, granules, powders, etc.; liquid formulations such as syrups, injections, etc.; injections, drops, suppositories, pessaryies. In some embodiments, compounds or pharmaceutical compositions in accordance with this disclosure may be administered by inhalation spray, nasal, vaginal, rectal, sublingual, or topical routes and may be formulated, alone or together, in suitable dosage unit formulations containing conventional non-toxic pharmaceutically acceptable carriers, adjuvants and vehicles appropriate for each route of administration.

A compound of the disclosure may be used to treat animals, including mice, rats, horses, cattle, sheep, dogs, cats, and monkeys. However, a compound of the disclosure may also be used in other organisms, such as avian species (e.g., chickens). One or more of the compounds of the disclosure may also be effective for use in humans. The term “subject” or alternatively referred to herein as “patient” is intended to be referred to an animal, such as a mammal (e.g., a human) that has been the object of treatment, observation or experiment. However, one or more of the compounds, methods and pharmaceutical compositions of the present disclosure may be used in the treatment of animals. Accordingly, as used herein, a “subject” may be a human, non-human primate, rat, mouse, cow, horse, pig, sheep, goat, dog, cat, etc. The subject may be suspected of having or at risk for having pain, anxiety, a mood disorder or depression.

An “effective amount” of a compound according to the disclosure may include a therapeutically effective amount or a prophylactically effective amount. A “therapeutically effective amount” refers to an amount effective, at dosages and for periods of time necessary, to achieve the desired therapeutic result, such as treatment of pain, anxiety, mood disorders or depression, or any condition described herein. A therapeutically effective amount of a compound may vary according to factors such as the disease state, age, sex, and weight of the individual, and the ability of the compound to elicit a desired response in the individual. Dosage regimens may be adjusted to provide the optimum therapeutic response. A therapeutically effective amount may also be one in which any toxic or detrimental effects of the compound are outweighed by the therapeutically beneficial effects. A “prophylactically effective amount” may refer to an amount effective, at dosages and for periods of time necessary, to achieve the desired prophylactic result, such as treatment of pain, anxiety, mood disorders or depression, or any condition described herein. Typically, a prophylactic dose may be used in subjects prior to or at an earlier stage of disease, so that a prophylactically effective amount may be less than a therapeutically effective amount. A suitable range for therapeutically or prophylactically effective amounts of a compound may be any integer from 0.1 nM-0.1 M, 0.1 nM-0.05 M, 0.05 nM-15 M or 0.01 nM-10 μM.

In alternative embodiments, in the treatment or prevention of conditions which may require treatment of pain, anxiety, mood disorders or depression, an appropriate dosage level may generally be about 0.01 to 500 mg per kg subject body weight per day and may be administered in single or multiple doses. In some embodiments, the dosage level may be about 0.1 to about 250 mg/kg per day. It will be understood that the specific dose level and frequency of dosage for any particular patient may be varied and may depend upon a variety of factors including the activity of the specific compound used, the metabolic stability and length of action of that compound, the age, body weight, general health, sex, diet, mode and time of administration, rate of excretion, drug combination, the severity of the particular condition, and the patient undergoing therapy.

It is to be noted that dosage values may vary with the severity of the condition to be alleviated. For any particular subject, specific dosage regimens may be adjusted over time according to the individual need and the professional judgement of the person administering or supervising the administration of the compositions. Dosage ranges set forth herein are exemplary only and do not limit the dosage ranges that may be selected by medical practitioners. The amount of active compound(s) in the composition may vary according to factors such as the disease state, age, sex, and weight of the subject. Dosage regimens may be adjusted to provide the optimum therapeutic response. For example, a single bolus may be administered, several divided doses may be administered over time or the dose may be proportionally reduced or increased as indicated by the exigencies of the therapeutic situation. It may be advantageous to formulate parenteral compositions in dosage unit form for ease of administration and uniformity of dosage. In general, compounds of the disclosure should be used without causing substantial toxicity, and as described herein, one or more of the compounds may exhibit a suitable safety profile for therapeutic use. Toxicity of a compound of the disclosure may be determined using standard techniques, for example, by testing in cell cultures or experimental animals and determining the therapeutic index. In some circumstances however, such as in severe disease conditions, it may be necessary to administer substantial excesses of the compositions. In some embodiments, one or more of the compounds disclosed herein may exhibit no observed toxicity at high doses, for example, 1000 μmol/kg.

In some embodiments, one or more of the compounds disclosed herein may exhibit enhanced or increased potency compared to an endogenous ENK peptide, such as Leu-ENK or Met-ENK. In some embodiments, one or more of the compounds disclosed herein may exhibit enhanced or increased potency compared to an ENK analogue. In some embodiments, one or more of the compounds disclosed herein may exhibit increased stability, for example, in plasma, compared to an endogenous ENK peptide, such as Leu-ENK or Met-ENK. In some embodiments, one or more of the compounds disclosed herein may exhibit increased stability compared to an ENK analogue.

In some embodiments, one or more of the compounds disclosed herein may exhibit increased membrane permeability compared to an endogenous ENK peptide, such as Leu-ENK or Met-ENK. In some embodiments, one or more of the compounds disclosed herein may exhibit increased membrane permeability compared to an ENK analogue. By “enhanced” or “increased” or “elevated” is meant an increase by any value between about 5% and about 90%, or of any value between about 30% and about 60%, or over about 100%, or an increase by about 1-fold, 2-fold, 5-fold, 10-fold, 15-fold, 25-fold, 50-fold, 100-fold, or more, in comparison to one or more of an endogenous ENK peptide, such as Leu-ENK or Met-ENK, or an ENK analogue. In some embodiments, by “enhanced” or “increased” or “elevated” stability, means stability in plasma for at least 5 hours, for example, 5 hours, 10 hours, 15 hours, 24 hours, 37 hours, 48 hours or more.

In some embodiments, one or more of the compounds disclosed herein may exhibit reduced or decreased side effects compared to an endogenous ENK peptide, such as Leu-ENK or Met-ENK. In some embodiments, one or more of the compounds disclosed herein may exhibit reduced or decreased tolerance, physical dependence, respiratory depression potential and/or constipation compared to an endogenous ENK peptide, such as Leu-ENK or Met-ENK. By “decreased” or “reduced” is meant a decrease by any value between about 5% and about 90%, or of any value between about 30% and about 60%, or over 100 about %, or a decrease by about 1-fold, 2-fold, 5-fold, 10-fold, 15-fold, 25-fold, 50-fold, 100-fold or more, in comparison to an endogenous ENK peptide, such as Leu-ENK or Met-ENK.

In some embodiments, one or more of the compounds disclosed herein may be used for treating pain, anxiety, mood disorders or depression.

In some embodiments, one or more of the compounds disclosed herein may be used in the manufacture of a medicament for treating pain, anxiety, mood disorders or depression.

In some embodiments, one or more of the compounds disclosed herein may be used in a method for treating pain, anxiety, mood disorders or depression, by (a) administering a compound as described herein or a pharmaceutical composition described herein to a subject in need of such treatment.

“Pain” as used herein includes, without limitation, acute pain, chronic pain, inflammatory pain, neuropathic pain, nociceptive pain, psychogenic pain, visceral pain, thermal pain, musculoskeletal pain, post-surgical pain, etc.

“Anxiety” as used herein refers to an emotion characterized by feelings of tension, worried thoughts and physical changes like increased blood pressure. Anxiety disorders or “anxiety” includes, without limitation, panic attacks, generalized anxiety disorder, social anxiety disorder (social phobia), specific phobias, separation anxiety disorder, etc.

“Mood disorders” refer to a general emotional state or mood that is distorted or inconsistent with a patient's circumstances and may interfere with the ability to function. Mood disorders include, without limitation, bipolar disorder, seasonal affective disorder (SAD), cyclothymic disorder, premenstrual dysphoric disorder, postpartum depression, persistent depressive disorder (dysthymia), disruptive mood dysregulation disorder, dysthymic disorder, depression related to medical illness, depression induced by substance use or medication, major depressive disorder or depression, etc.

In some embodiments, one or more of the compounds disclosed herein may be provided in a commercial package including (a) a compound as described herein; and (b) instructions for the use of such a compound for treating pain, anxiety, mood disorders or depression. In some embodiments, one or more of the compounds provided in the commercial package may be in the form of a pharmaceutical composition.

The present invention will be further illustrated in the following examples.

### EXAMPLES

Materials and Methods

Synthesis Methods

The structure and general synthesis adopted for preparing Leu-ENK analogues are outlined in FIG. 2. The synthesis of Leu-ENK analogues was achieved starting from glycylglycine 11. In the first step (A), the free amine of 11 was protected with Boc anhydride and the resulting N-Boc-glycylglycine 12 was conjugated with Methyl L-phenylalanine (B) in the second step using EDC-HCl, and 4-DMAP to generate intermediate 13, which was converted to intermediate 14 by ester hydrolysis in the presence of LiOH and THF at room temperature (C). The compound was treated with methyl L-leucine in the presence of EDC-HCl, 4-DMAP, and THF (D). The resulting Boc-tetrapeptide 15 was deprotected with TFA in the presence of DCM to produce the intermediate compound 16 (E). L-Tyrosine was conjugated with different aryl, alkyl and acetyl chlorides in the presence of EDC-HCl, 4-DMAP, and THF and subsequently reacted with intermediate 16 in the presence of DCC, HOBt, DIPEA, and DMF (Fa, Fb). The resulting Leu-ENK analogues 2, 3, 4, 5, 6, 7, 8, and 9 contained different N-acyl, N-alkyl and N-benzoyl modifications and a C-terminal methyl ester. Subsequent ester hydrolysis of analogues 2, 3, 4, 5, 6, 7, 8, and 9 in the presence of LiOH was performed to produce compounds with a C-terminal carboxylic acid.

DOR Binding Assay

Binding studies were performed by Eurofins Scientific (Cereb, France). Briefly, COS-1 cells transfected with plasmids encoding human DOR (Gene ID: 4985) were grown in Dulbecco's modified Eagle's medium with 10% fetal calf serum and scraped from plates. Membranes were prepared using a Dounce homogenizer and stored at −80° C. until used. Membranes (40 μg protein/tube) were suspended in Tris-HCl, pH 7.4, for 2 h at 25° C., rapidly filtered on 0.1% polyethylenimine-pretreated GF/B filters and washed with the same cold Tris buffer (3×) using a Brandel cell harvester. Displacement of radioligand [³H]D-Ala2-D-Leu⁵-enkephalin (DADLE, 0.5 nmol/L) with peptides (0.6 nmol/L) was measured after incubation (60 min, room temperature) using a scintillation counter. Nonspecific binding was determined in the presence of naltrexone (10 μmol/L). DOR binding ability was expressed as relative scintillation compared to native Leu-ENK.

Plasma/CSF Stability Assay

Peptides (1.5 g/L) dissolved in saline/TWEEN 80/DMSO (90:5:5, v/v) were mixed with mouse plasma or human cerebrospinal fluid, respectively, to a final concentration of 0.315 mmol/L and incubated in a thermomixer (37° C., 750 rpm) Aliquots (45 μL) were mixed with aqueous acetonitrile (90%, v/v, 300 μL) containing formic acid (FA, 0.1%, v/v) and incubated on ice (30 min). After two centrifugation steps (12,000×g, 5 min), the supernatant (300 μL) was diluted in water (900 μL), dried by lyophilization, reconstituted in aqueous acetonitrile (4.5%, v/v) containing FA (0.1%, v/v), stored at −20° C., and analyzed with reverse-phase chromatography (RPC) on a C8-column (1 mm×150 mm) with a linear aqueous acetonitrile gradient typically from 5 to 95% eluent B in 15 min (eluent A: 0.1% (v/v) aqueous FA; eluent B: 90% aqueous acetonitrile containing 0.1% (v/v) FA. The RPC was coupled on-line to a UV detector measuring the absorbance at 214 nm and a mass spectrometer (MS) for peak identification. Peptide quantities were estimated using peak areas of the UV signal relative to the initial peak areas (t=0).

Animals

Experiments involved adult female CD-1 mice (25-30 g) housed under a 12 h light/dark cycle and had ad libitum access to rodent chow and water. Mice were allowed an acclimatization period of at least one week in the animal facility before experiments. Injections of drugs or vehicle were administered s.c or i.p. into animals at a volume of 10 mL/kg. In the formalin foot assay, each mouse was used once and euthanize immediately upon completion of the experiment due to irreversible tissue damage in the injected paw. For the Forced Swim Test, Tail Suspension Test and Open Field test, the experiments were performed during the light phase between 9 AM-5 PM.

Drug Delivery

Compounds were dissolved in saline/TWEEN 80 (9/1, v/v) and either subcutaneously (s.c.) injected or delivered intranasally (i.n.) to female CD1 mice. For s.c. injection, the KK-103 dose was fixed at 20 μmol/kg (equivalent to 13 mg/kg). For i.n. delivery, KK-103 was delivered to mice 4 times at 6 μl/nasal cavity for a total combined dose of 2 μmoL/kg.

For the Forced Swim Test, Tail Suspension Test and Open Field test, injections of drugs or vehicle were administered to animals subcutaneously (s.c.) or intraperitoneally (i.p.) at a volume of 10 mL/kg. Solutions were prepared freshly the day prior to the experiment by dissolving drugs in a mixture of saline, Tween 80 and DMSO at a volume ration of 18:1:1. Vehicle and KK-103 (15 mg/kg) were injected 120 min and Desipramine hydrochloride (Sigma-Aldrich, Mississauga, ON, Canada) (30 mg/kg, i.p.) 30 min before testing. All injections were administered i.p. except for KK-103 which was injected s.c.

Hot Plate Test

The antinociceptive activity was studied on female CD-1 mice using a hot-plate test after s.c. injection of compounds listed in Table 1 at a dose of (20 μmol/kg) or morphine at a dose of 10 mg/kg. In some studies, mice received KK-103 (13 mg/kg) that was co-delivered with either naloxone (4 mg/kg, i.p.) or methylnaloxone (4 mg/kg, i.p.). In other studies, groups of mice received either KK-103 (13 mg/kg) or morphine (10 mg/kg) twice-daily for 7 days and the analgesic activity was measured on day 1, 4 and 7. The latency of response (LR) to a heated surface (52° C.) was observed and The Percentage Maximum Possible Effect (% MPE) was calculated as the percentage difference between the measured response (LR) and the baseline response (BR) divided by the difference between the maximum response (MR) and the baseline response (BR) using equation 1. MR was set as 30 s to avoid heat damage to mice paws and BR was determined in individual mouse before the experiment.

$$\% MPE = \frac{LR - BR}{MR - BR} \cdot 100 \qquad ( 1 )$$

The Area under the curve (AUC_{0-5 h}) was calculated considering the % MPE within a duration of 5 h.

Dependence Test

Female CD1 mice received twice-daily treatments of KK-103 (20 μmol/kg equivalent to 13 mg/kg, s.c.) or morphine (10 mg/kg, s.c.) for seven days. On day 7, 2 hrs after the final dose, mice received naloxone (4 mg/kg, intraperitoneal (i.p.)) and the number of jumps within 10 min were counted.

Toxicity

Female CD1 mice received the highest deliverable single dose of KK-103 (1000 μmol/kg, s.c.) limited by solubility of the compound (5 g/L) in normal saline/TWEEN 80 (9/1, v/v) and the maximal injection volume of 3 mL. Animals were monitored and evaluated every 30 min. After 3 h, the time of maximal antinociceptive effect, animals were euthanized.

Formalin Foot Assay

On the day of the experiment, mice were acclimatized to the experimental setup by putting them in darkened, non-transparent plexiglas cylinders (height, 20 cm; diameter, 9 cm) placed on a glass plane for 2 h. Mice received morphine (10 mg/kg, s.c.) or vehicle (s.c.) 5 min, and KK-103 (13 mg/kg, s.c.) 150 min before injection of 20 μL of a 2.5% formalin solution into the intraplantar surface of the right hind paw using a 29G 0.5 mL insulin syringe (BD, Mississauga, ON, Canada). After successful injection, mice were immediately placed into cylinders and their behavior was monitored for 1 h. Video cameras (Lorex, ON, Canada) underneath each animal were used to record the nociceptive responses defined as licking or biting at the formalin injected right paw. The accumulated licking and biting time of each animal was determined and recorded in 5 min bins. The total response time was plotted as early phase 1 from 0 to 5 min, and late phase 2 from 20 to 40 min after formalin injection. Similarly, for the dose response relationship doses of KK-103 (vehicle, 5, 10, 20, 50 mg/kg) were administered s.c. 150 min prior to formalin injection. Accumulated licking and biting times were recorded and analyzed as stated above.

Respiratory Rate Assay

The respiratory rate was assessed in awake mice lightly restrained in a well-ventilated 50 mL falcon tube with a darkened tip. Prior to the drug treatment, each animal was habituated in the tube for 30 min. The falcon tube was secured onto a stand facing lengthwise to a video camera. Animals (n=6) were randomly assigned to receive an s.c. injection of either vehicle control, morphine (10 mg/kg) or KK-103 (13 mg/kg). Animals were recorded at selected time points for 10 min to measure the respiration rate.

Blood Sampling and Plasma Sample Preparation

A sterile filtered solution (0.22 μm membrane syringe filter) of KK-103 was administered either via subcutaneous or intravenous injection into female CD-1 mice. Blood collection from mice was performed by saphenous vein punctuation (volume withdrawn <7 mL/kg) followed by a cardiac puncture later, allowing for 2 sampling time points per animal with a minimum sampling interval of 50 min. Cardiac puncture was an experimental endpoint after which animals were euthanized using isoflurane followed by cervical dislocation. Blood samples were collected in EDTA coated collection tubes and chilled on ice immediately after collection. Blood plasma was separated from whole blood by centrifugation at 2.000 g for 7 min and carefully removed from the cell pellet. Blood plasma samples were immediately frozen on dry ice and stored at −80 C until further analysis.

Samples for liquid chromatography-mass spectrometry analysis were prepared by acetonitrile protein precipitation. Therefore, 50 μL of plasma was combined with 300 μL ice cold 90% acetonitrile containing 0.1% formic acid, briefly vortexed and put on ice for 30 min. After that samples were centrifuged at 4 C for 5 min (>12 k rpm) and supernatant (325 μL) transferred in a new tube. The previous centrifugation step was repeated and a final volume of 305 μL of the supernatant transferred into a new 1.5 mL tube. Water (800 μL) was added to samples after which they were frozen and lyophilized. The dried samples were resolubilized in 50 μL 4.5% acetonitrile, 0.1% formic acid in water, vortexed (1 min), sonicated (5 min), vortexed (1 min) and centrifuged for 5 min (>12 k rpm). Samples were measured in an ACQUITY UPLC H-Class chromatography system (Waters, Milford, MA) and a sample volume of 10 μL was injected onto the reverse phase column.

UPLC

An ACQUITY UPLC H-Class chromatography system (Waters, Milford, MA) coupled on-line to a photodiode array detector (absorbance recorded at 214 nm, Waters) and to the electrospray ionization source on a mass spectrometer (QDa detector, Waters) operated in the positive ion mode was used. Separation relied on a BEH-C18 column (inner diameter: 1.1 mm; length: 50 mm; particle size: 1.7 μm, Waters) at a flow rate of 0.1 mL/min using a linear aqueous acetonitrile gradient in the presence of FA (0.1%, v/v).

Ionization was carried out at a source temperature of 450° C. using a cone voltage of 10 V and a capillary voltage of 1.5 kV. Mass spectra were typically acquired for an m/z range from 50 to 1250 at a sampling rate of 2 points per second. Quantities of the peptide were determined based on a calibration curve of the analyte in mouse plasma and the peak areas of the UV signal at 214 nm as well as mass spectrometry by detecting the singly charged molecular ions.

Statistical Analysis

Data presentation and statistical analysis are as described herein. Statistical analysis was conducted using GraphPad Prism software (GraphPad Software, San Diego, CA). A difference with P<0.05 was considered statistically significant.

Forced Swim Test

The experimental setup consisted of a transparent cylindrical glass container with a height of 193 mm and a diameter of 131 mm that was placed between black Plexiglass dividers and filled with water (24±0.1° C.) to approximately 8 cm below the rim. Mice naïve to the test apparatus were brought into the laboratory space in their home cages and were acclimatized to the room for at least 1 hour. The test room was indirectly illuminated by diffuse overhead white light during acclimation and testing. Each trial was started by carefully placing mice into the center of the cylinder to allow it to swim freely. Mice were kept in the water for 6 minutes, which were started as soon as the mouse touched the water. After the 6-minute trial the animal was removed from the tank, dried with a paper towel, placed in a recovery cage under a heat lamp for approximately 10 min and then returned to the home cage. The water in the cylinder was replaced for each animal. The entire test was recorded with a video camera (Canon VIXIA HF R800 HD), placed approximately 0.6 m in front of the cylinder, and the last 4 minutes of each trial were analyzed by blinded observers to obtain behavioral scores. Immobility was determined indirectly by subtracting the total mobility time from the analyzed trial duration (Immobility=240 s−Mobility(s)). Mobility was defined as “any movements other than those necessary to balance the body and keep the head above the water”⁵⁶. Each trial was observed in duplicate by at least 2 trained, independent observers and the mean of scores was used.

Tail Suspension Test

Mice naïve to the experimental setup were brought into the laboratory space in their home cages and were acclimatized to the room for at least 1 hour. The test room was indirectly illuminated by diffuse overhead white light during acclimation and testing. Each trial was started by suspending mice by their tail to a bar about 60 cm above a bench top. Conventional electrical tape was used to adhere the tail approximately 1.5 cm from the tip to the surface of the bar. A plastic cylinder of 4 cm length and 1 cm diameter around the tail was used to prevent mice from climbing up their tail and reach the bar. Each trial lasted for 6 min from the moment of suspension and was recorded with a video camera (Canon VIXIA HF R800 HD), placed approximately 1 m in front of the suspended animal. After the trial the mouse was immediately removed from the suspension attachment and returned to its home cage. Behavioral scores were obtained by analyzing all 6 minutes of the trial by blinded observers. Immobility was determined indirectly by subtracting the total mobility time from the analyzed trial duration (Immobility=360 s−Mobility(s)). Mobility was defined as movements include trying to reach the suspension attachment, strong shaking of the body, and running attempts. Small movements confined to the front legs, oscillations, and pendulum like swings are not counted as mobility 57,58. Each trial was observed in duplicate by at least 2 trained, independent observers and the mean of scores was used.

Open Field Test

A Plexiglas open field arena measuring 60×60×40 cm with a camera (Canon VIXIA HF R800 HD) mounted vertically approximately 2 m above the center of the arena was used. The setup was situated in a room indirectly illuminated by diffuse white light separate from the housing room. The open field test was performed with mice that were exposed to the FST or TST 48 h prior but received a vehicle or drug injection at the same day following re-randomization of treatment. Each trial was started by placing mice into the center of the arena in which the animal's movement was recorded for 8 minutes. After the trial the mouse was immediately removed from the area and returned to its home cage and the box cleaned with 70% ethanol. Behavioral analysis was carried out using the video tracking software Smart v3.0.06 (Panlab Harvard Apparatus, Saint-Laurent, QC, Canada) which reported locomotor activity as total distance during the first 6 min of each trial.

### Example 1: Binding of Leu-ENK Derivatives to Delta Opioid Receptors

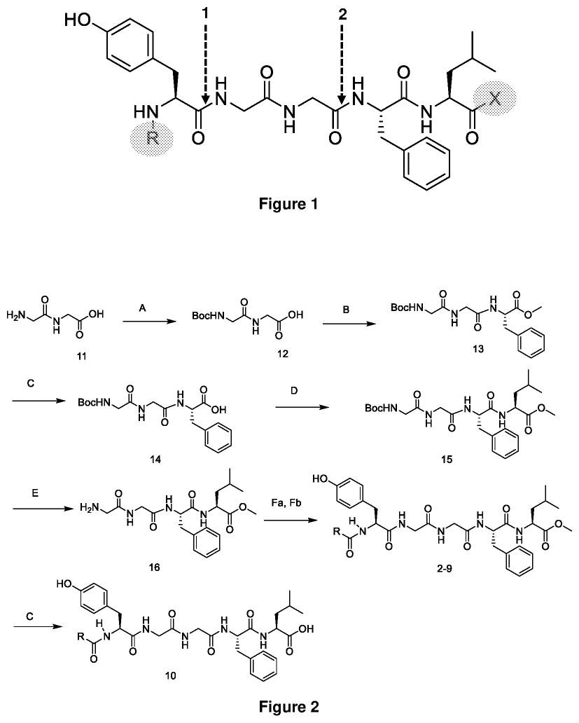

Native Leu-ENK was modified with a set of eight hydrophobic moieties (FIG. 1, ‘R’) at the N-terminus to generate a library of Leu-ENK derivatives, all containing a C-terminal methyl ester (X: OMe). The DOR binding ability of each derivative was measured as relative scintillation compared to native Leu-ENK in a cell-based radioligand displacement assay using DADLE. Minor modifications on N- and C-terminus slightly reduced but did not abolish the binding with DOR (Table 1). Among the conjugates, KK-102 (compound 6) displayed the strongest binding for DOR (˜70% relative to Leu-ENK). KK-102 and KK-103 (compound 10) were also compared. DOR binding of these compounds did not differ significantly (KK-102: 68%, KK-103: 65%).

TABLE 1
Comparison of native Leu-ENK and Leu-ENK conjugates containing a C-
terminal ester (X: OMe) but different N-terminal (R).

| | | | | Intact | |
|---|---|---|---|---|---|
| | | | Relative | peptide | |
| | | | Binding | after 1 h | Antinociception, |
| | Compound | N-terminal | to DOR | in plasma | AUC₀₋₅ₕ |
| No | Code | Modification (R)^{a} | [%]^{b} | [%] | [% MPE•h]^{c} |
| 1 | Leu-ENK | 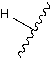 [SMILES: *[H]] | 100 | 23 ± 3 | 14 ± 6 |
| 2 | KK-14 | 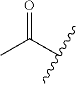 [SMILES: *C(C)=O] | 38 | 82 ± 3 | 88 ± 10 |
| 3 | KK-81 | 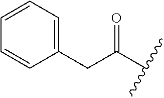 [SMILES: *C(=O)Cc1ccccc1] | 41 | 88 ± 11 | 37 ± 10 |
| 4 | KK-82 | 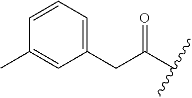 [SMILES: *C(=O)Cc1cccc(C)c1] | 49 | 51 ± 9 | 43 ± 17 |
| 5 | KK-93 | 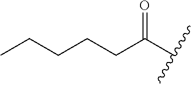 [SMILES: *C(=O)CCCCC] | 33 | 85 ± 11 | 51 ± 13 |
| 6 | KK-102/ KK-103 | 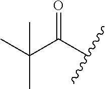 [SMILES: *C(=O)C(C)(C)C] | 68 | 93 ± 1 | 142 ± 15 |
| 7 | KK-105 | 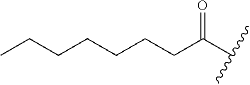 [SMILES: *C(=O)CCCCCCC] | 59 | 80 ± 12 | 75 ± 22 |
| 8 | KK-108 | 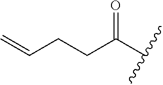 [SMILES: *C(=O)CCC=C] | 34 | 73 ± 7 | 48 ± 15 |
| 9 | KK-112 | 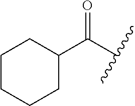 [SMILES: *C(=O)C1CCCCC1] | 13 | 62 ± 7 | 51 ± 10 |

^{a}N-terminal modification of R as shown in FIG. 1,
^{b}The binding to human DOR measured as relative scintillation compared to Leu-ENK at 6 nmol/L in a cell-
based competitive radioligand displacement assay. Values represent mean of duplicates.
^{c}The antinociceptive activity in female CD1 mice is expressed as the area under the curve (AUC) of the
antinociceptive activity plot (percent maximal possible effect (% MPE) vs time) using a hot plate test after
subcutaneous (s.c.) injection of compounds. Compounds were dissolved in saline containing 10% TWEEN
80 and compared at a matched dose of 20 μmol/kg. % MPE = (LR-BR)/(MR-BR) × 100%, where LR, MR and
BR represent measured, maximum (30 s) and baseline (~5 s) response time, respectively.
Data = mean ± SEM, n = 6.

### Example 2: In Vitro Stability and Pharmacology of Leu-ENK Derivatives

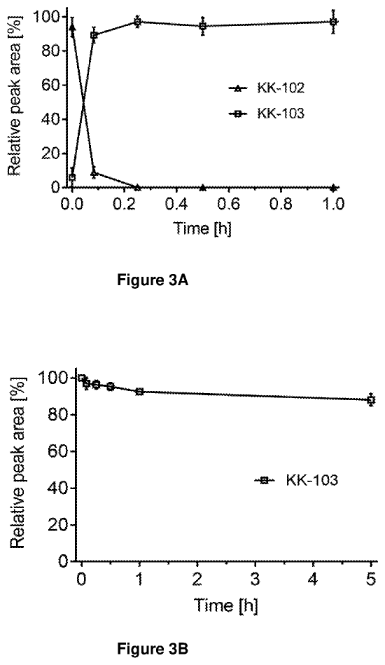

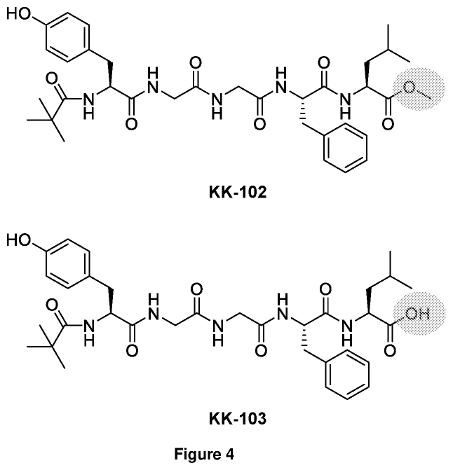

Among the derivatives, KK-103 exhibited the highest stability in plasma. When incubated in mouse plasma, KK-102 lost its C-terminal modification converting to KK-103, i.e. deesterification of the methyl ester to carboxylic acid (FIG. 3A). After 1 h incubation with mouse plasma, 93% of KK-103 remained intact (FIG. 3B). KK-103 was stable for at least 5 hours in mouse plasma (FIG. 3B). Based on the plasma stability results, KK-103 appears to be the active compound in both cases. The structures of KK-102 and KK-103 are depicted in FIG. 4.

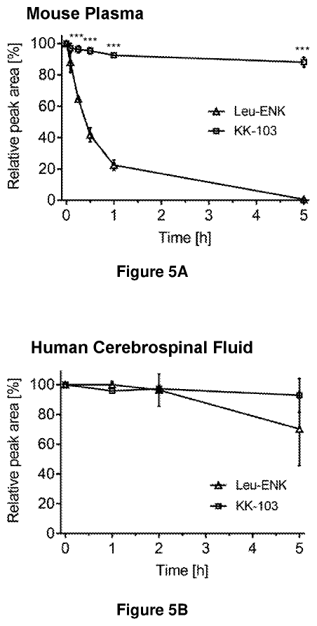

KK-103 was more stable than Leu-ENK in mouse plasma (half-lives of 37 h vs 25 min) (FIG. 5A), as well as in cerebrospinal fluid, where KK-103 showed almost no degradation after 5 h of incubation (FIG. 5B). Additionally, KK-103 was stable in the presence of human liver microsomes (<10% drug degradation after 1 h inhibition) and did not inhibit human liver microsome enzymes, suggesting low liver metabolism and low potential for drug-drug interaction for KK-103.

Blood Plasma Concentrations of KK-103 after Subcutaneous or Intravenous Injection

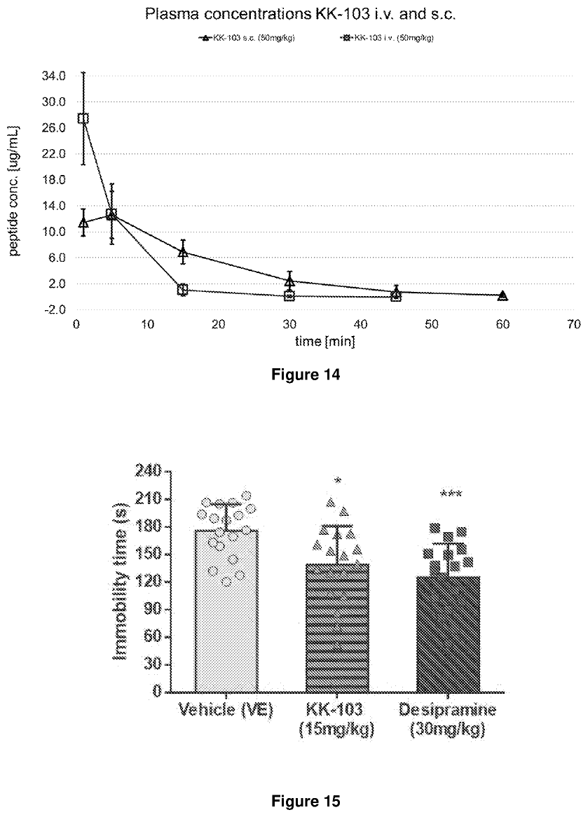

The plasma concentrations of KK-103 was monitored over time in female CD-1 mice following the administration of KK-103 (50 mg/kg) by either s.c. or i.v. delivery. As shown in FIG. 14, KK-103 was cleared from the circulation with elimination half-lives of 4.8 and 8.9 minutes for i.v. and s.c. route, respectively.

### Example 3: Antinociceptive Activity of Leu-ENK Derivatives In Vivo

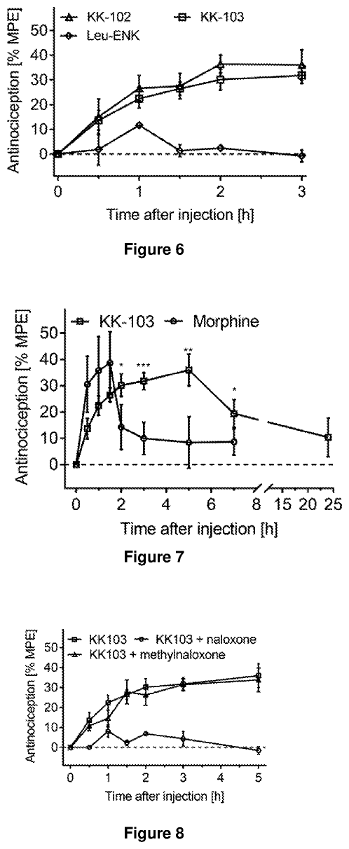

Since conversion of KK-103 to Leu-ENK was slow in physiological fluids and KK-103 exhibited relatively strong binding to DOR (˜70% relative to Leu-ENK, Table 1), these results suggest that KK-103 works as an active agent in vivo, rather than as a prodrug. In comparison to Leu-ENK, KK-102 and KK-103 both showed an improved analgesic activity in mice using a hot-plate study (FIG. 6). Leu-ENK displayed little antinociceptive activity in mice under the hot-plate test, while KK-103 achieved an analgesic effect (15-20% maximal possible effect, MPE) in 30 min after s.c. injection. The dose was fixed at 20 μmol/kg and compounds were dissolved in saline containing 10% TWEEN 80. The effect of KK-103 reached maximum at 2 h (˜30% MPE) and plateaued for at least another 3 h. The effects of other conjugates are reported in Table 1.

A dose of 20 μmol/kg KK-103 showed an antinociceptive effect similar to 10 mg/kg morphine (FIG. 7). In the hot-plate test, morphine (10 mg/kg) rapidly achieved its maximal effect (30-40% MPE) within 15 min after s.c. injection but showed activity loss at 2 h, while KK-103 at 20 μmol/kg (equivalent to 13 mg/kg) started producing analgesic activity 30 min post s.c. injection. The analgesic effect of KK-103 increased over time and reached its maximum effect (30% MPE) at 2 h and then plateaued until 5 h, followed by a return to baseline within 24 h. Co-administration with the exclusively peripherally acting opioid receptor antagonist methylnaloxone did not decrease the activity of KK-103, while co-administration with globally acting antagonist naloxone abolished it completely (FIG. 8). These data indicate that KK-103 acted in the CNS for its activity in this model, a notion further supported by the significant concentrations of KK-103 detected by ultra-performance liquid chromatography (UPLC) in the plasma (170 μg/g) and brain (0.2 μg/g) 2 h after s.c. injection.

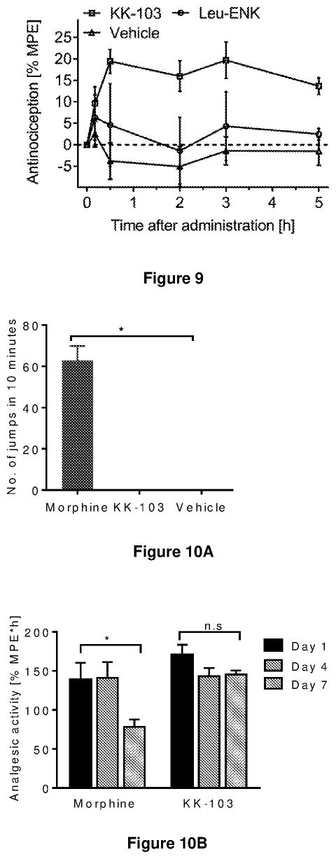

Non-parenteral administration, such as oral and intranasal (i.n.), offers dosing convenience and improved medication adherence with low risk of infection compared to parenteral delivery, including i.v. and s.c. and it is less expensive to manufacture non-parenteral dosage forms. KK-103 was dissolved in saline/TWEEN 80 (9/1 v/v) and i.n. delivered to mice (6 μL/nasal cavity×4 times for a total combined dose of 2 μmol/kg), producing a significant analgesic effect (15-20% MPE) in female CD1 mice in the hot plate test, while Leu-ENK displayed minimal activity (FIG. 9). The effect of i.n. KK-103 at 2 μmol/kg was only slightly lower than s.c. injection at 20 μmol/kg.

### Example 4: Dependence Effect and Toxicity of Leu-ENK Derivatives In Vivo

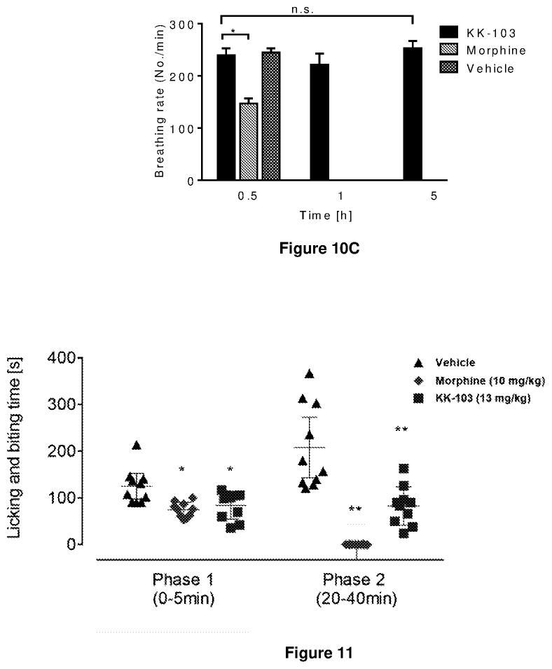

The equipotent doses for KK-103 and morphine were 20 μmol/kg and 10 mg/kg, respectively (FIG. 7). At the highest deliverable dose of 1000 mmol/kg, KK-103 induced no toxicity, while morphine is only safe to use up to 60 mg/kg. KK-103 did not induce breathing depression, while morphine-treated mice showed significant reduction of breathing rate after 30 min post injection (FIG. 10C). The therapeutic window for KK-103 was significantly broader compared to morphine and unlike morphine, long-term use of KK-103 did not induce any opioid withdrawal symptoms in mice (FIG. 10). We compared the development of physical dependence of KK-103 with morphine. Female CD-1 mice were injected twice daily with morphine (10 mg/kg), KK-103 (13 mg/kg), or vehicle for eight days. After the last treatment, withdrawal, which in mice is associated with jumping behavior, was induced by an i.p. dose of naloxone (Nlx) at 4 mg/kg. The number of jumps of each mouse in the first 10 min post Nlx injection was measured (FIG. 10A). As shown in FIG. 10A, repeated administrations of morphine-induced significant withdrawal syndromes in mice following Nlx challenge, indicating opioid dependence. The group treated with KK-103, on the other hand, did not show any signs of physical dependence, i.e. not a single jump was observed. Tolerance is another common side effect of chronic opioid use and involves multiple mechanisms that primarily result in a gradual loss of analgesic potency and efficacy. Therefore, we investigated whether mice developed tolerance to KK-103 and morphine after chronic use. The development of antinociceptive tolerance was assessed following s.c. administrations of morphine (10 mg/kg) or KK-103 (13 mg/kg) twice daily for 7 consecutive days. As shown in FIG. 10B, the overall antinociceptive activity of morphine on day 4 remained unchanged compared to day 1 (123 vs 143% MPE-h, respectively) but was significantly decreased by 35% on day 7 (80% MPE-h), indicating the development of tolerance on day 7. In contrast, KK-103 remained equally efficacious after 7 days of treatments (170 vs 145% MPE-h, day 1 and day 7, respectively), indicating no development of tolerance. Overall, KK-103 showed no toxicity and a lethal dose could not be administered.

### Example 5: Analgesic Effect of KK-103 Compared to Morphine

The formalin injection model was employed to compare the analgesic activity of KK-103 with morphine. This test uses a chemical noxious stimulus, which produces a longer lasting and progressing nociception. The measured parameter is the cumulative time that rodents spend licking or biting the formalin-injected paw in response to the stimulus.⁴⁷ The behavior of animals after intraplantar injection of formalin is biphasic, resulting from direct stimulation of nociceptors in the first phase and inflammatory response in the second phase.⁴⁸

As shown in FIG. 11 the paw licking and biting time in phase 1 from 0 to 5 min (FIG. 11), after morphine administration was reduced by 30%, and 25% in KK-103 treated mice compared to the control. In phase 2, KK-103 significantly reduced the licking and biting time to ˜50% of the mean response of the vehicle group, while morphine completely diminished the nociceptive response in all mice. While both morphine and KK-103 were effective in both phases, the phase 2 effect appeared to be more significant than that in phase 1 for both drugs. These results clearly show the effectiveness of KK-103 in this inflammation-related pain model.

### Example 6: Dose Response Relationship of KK-103 in the Formalin Foot Model

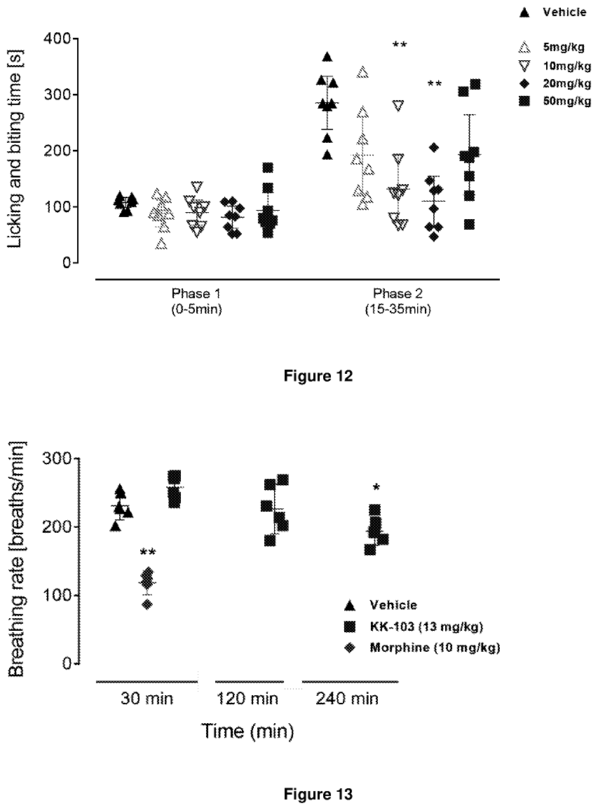

Next, we examined the dose-dependent efficacy of KK-103. KK-103 was administered s.c. at four different doses ranging from 5 mg/kg to 50 mg/kg, and the antinociceptive effect was assessed in the formalin foot as described above. As shown in FIG. 12, the effect of KK-103 increased with an increase of the dose and reaches a maximum effect at 20 mg/kg, with a significantly reduced average licking and biting time by roughly 60% compared to the mean response of the vehicle group in phase 2. A further increase of dosage to 50 mg/kg did not result in a further increase of antinociceptive efficacy of KK-103.

### Example 7: Effect of KK-103 on Respiratory Rate Compared to Morphine

To characterize the safety of KK-103, we compared its effect on the respiratory rate of mice and compared it to the respiratory depressive effects of morphine. Breathing suppression is the leading cause of death after acute morphine poisoning and significantly limits the dose that can be safely administrated.⁴⁹ Therefore, a significant reduction or suppression of the breathing rate is considered the most concerning and dangerous side effect of opioids.⁴⁹ Animals (n=6) were randomly assigned to receive either vehicle, KK-103 (13 mg/kg) or morphine (10 mg/kg) via s.c. injection, after which their breathing rates were measured. As shown in FIG. 13, morphine significantly decreased the respiratory rate by 56% compared with the vehicle control at 30 min post injection, which is in line with previous reports.^{50,51} In contrast, the respiration rate of KK-103-treated mice was comparable to that of mice treated with vehicle at any time point between 30 and 240 min.

### Example 8: Antidepressant Efficacy of KK-103

To investigate whether KK-103 exerted antidepressant activity in mice we used the forced swim test (FST) and the tail suspension test (TST), two experimental approaches which are commonly used to evaluate antidepressant drugs. In both tests animals were exposed to stress, which effects the tendency for major depression⁵⁵. In these behavioral tests, the immobility of animals, or giving up on escape related behaviors, was measured, which is considered to reflect behavioral despair that is indicative of depression⁵⁶⁻⁵⁷. Finally, the open field test was used as a control assay to confirm whether treatments could affect the general locomotor function of animals and introduce conflicting results to FST and TST. For example, if a drug increases walking distance in the open field test, then its effect in reducing the immobility time in FST and TST cannot be concluded as antidepression efficacy.

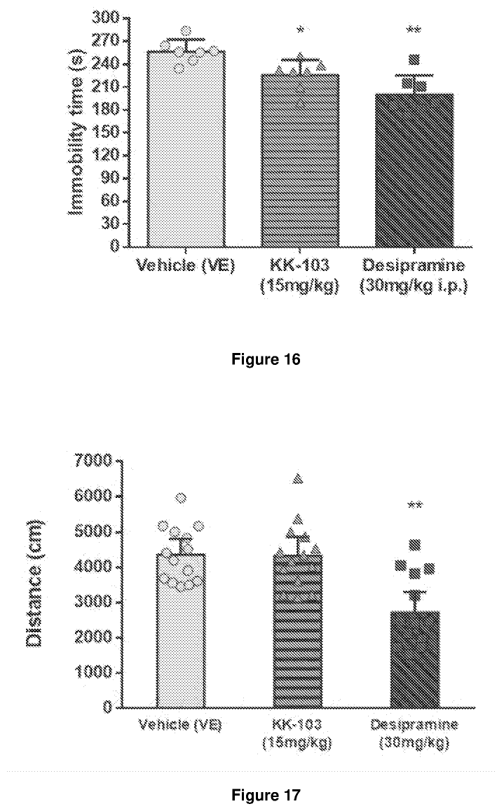

The results (FIGS. 15 and 16) suggested that KK-103 shows significant antidepressant effect (i.e. desipramine is a positive control) in the mice models without effecting the locomotor activity (FIG. 17).

All citations are hereby incorporated by reference.

The present invention has been described with regard to one or more embodiments. However, it will be apparent to persons skilled in the art that a number of variations and modifications can be made without departing from the scope of the invention as defined in the claims. Therefore, although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. It is to be understood that specific embodiments may be combined in any manner and in any number to create additional embodiments and any permutations and combinations of the embodiments should be considered disclosed by the description of the present application unless the context indicates otherwise. Numeric ranges are inclusive of the numbers defining the range. Recitation of numeric ranges of values herein is merely intended to serve as a shorthand method of referring individually to each separate value falling within the range. Unless otherwise indicated herein, each individual value is incorporated into the specification as if it were individually recited herein. The terms “a” and “an” and “the” and similar reference used in the context of describing the invention are to be construed to cover both the singular and the plural, unless otherwise indicated herein or clearly contradicted by context. In the description, the word “comprising” is used as an open-ended term, substantially equivalent to the phrase “including, but not limited to,” and the word “comprises” has a corresponding meaning. It is to be however understood that, where the words “comprising” or “comprises,” or a variation having the same root, are used herein, variation or modification to “consisting” or “consists,” which excludes any element, step, or ingredient not specified, or to “consisting essentially of” or “consists essentially of,” which limits to the specified materials or recited steps together with those that do not materially affect the basic and novel characteristics of the claimed invention, is also contemplated. Citation of references herein shall not be construed as an admission that such references are prior art to the present invention. All publications are incorporated herein by reference as if each individual publication was specifically and individually indicated to be incorporated by reference herein and as though fully set forth herein. The invention includes all embodiments and variations substantially as hereinbefore described and with reference to the examples and drawings.

### REFERENCES

1. Pasternak, G. W.; Pan, Y. X., Mu opioids and their receptors: evolution of a concept. Pharmacol Rev 2013, 65 (4), 1257-317.
2. Pradhan, A. A.; Befort, K.; Nozaki, C.; Gaveriaux-Ruff, C.; Kieffer, B. L., The delta opioid receptor: an evolving target for the treatment of brain disorders. Trends Pharmacol Sci 2011, 32 (10), 581-90.
3. Williams, J. T.; Christie, M. J.; Manzoni, O., Cellular and synaptic adaptations mediating opioid dependence. Physiol Rev 2001, 81 (1), 299-343.
4. Benyamin, R.; Trescot, A. M.; Datta, S.; Buenaventura, R.; Adlaka, R.; Sehgal, N.; Glaser, S. E.; Vallejo, R., Opioid complications and side effects. Pain Physician 2008, 11 (2 Suppl), S105-20.
5. Godfrey, L.; Iannitelli, A.; Garrett, N. L.; Moger, J.; Imbert, I.; King, T.; Porreca, F.; Soundararajan, R.; Lalatsa, A.; Schatzlein, A. G.; Uchegbu, I. F., Nanoparticulate peptide delivery exclusively to the brain produces tolerance free analgesia. J Control Release 2018, 270, 135-144.
6. Sitbon, P.; Van Elstraete, A.; Hamdi, L.; Juarez-Perez, V.; Mazoit, J. X.; Benhamou, D.; Rougeot, C., STR-324, a Stable Analog of Opiorphin, Causes Analgesia in Postoperative Pain by Activating Endogenous Opioid Receptor-dependent Pathways. Anesthesiology 2016, 125 (5), 1017-1029.
7. Corbett, A. D.; Henderson, G.; McKnight, A. T.; Paterson, S. J., 75 years of opioid research: the exciting but vain quest for the Holy Grail. Br J Pharmacol 2006, 147 Suppl 1, S153-62.
8. Land, B. B.; Bruchas, M. R.; Lemos, J. C.; Xu, M.; Melief, E. J.; Chavkin, C., The dysphoric component of stress is encoded by activation of the dynorphin kappa-opioid system. J Neurosci 2008, 28 (2), 407-14.
9. Vanderah, T. W.; Largent-Milnes, T.; Lai, J.; Porreca, F.; Houghten, R. A.; Menzaghi, F.; Wisniewski, K.; Stalewski, J.; Sueiras-Diaz, J.; Galyean, R.; Schteingart, C.; Junien, J. L.; Trojnar, J.; Riviere, P. J., Novel D-amino acid tetrapeptides produce potent antinociception by selectively acting at peripheral kappa-opioid receptors. Eur J Pharmacol 2008, 583 (1), 62-72.
10. Arendt-Nielsen, L.; Olesen, A. E.; Staahl, C.; Menzaghi, F.; Kell, S.; Wong, G. Y.; Drewes, A. M., Analgesic efficacy of peripheral kappa-opioid receptor agonist CR665 compared to oxycodone in a multi-modal, multi-tissue experimental human pain model: selective effect on visceral pain. Anesthesiology 2009,111 (3), 616-24.
11. Vanderah, T. W., Delta and kappa opioid receptors as suitable drug targets for pain. Clin J Pain 2010, 26 Suppl 10, S10-5.
12. Kiritsy-Roy, J. A.; Marson, L.; Van Loon, G. R., Sympathoadrenal, cardiovascular and blood gas responses to highly selective mu and delta opioid peptides. J Pharmacol Exp Ther 1989, 251 (3), 1096-103.
13. May, C. N.; Dashwood, M. R.; Whitehead, C. J.; Mathias, C. J., Differential cardiovascular and respiratory responses to central administration of selective opioid agonists in conscious rabbits: correlation with receptor distribution. Br J Pharmacol 1989, 98 (3), 903-13.
14. Pfeiffer, A.; Feuerstein, G.; Kopin, I. J.; Faden, A. I., Cardiovascular and respiratory effects of mu-, delta- and kappa-opiate agonists microinjected into the anterior hypothalamic brain area of awake rats. J Pharmacol Exp Ther 1983, 225 (3), 735-41.
15. Shook, J. E.; Pelton, J. T.; Hruby, V. J.; Burks, T. F., Peptide opioid antagonist separates peripheral and central opioid antitransit effects. J Pharmacol Exp Ther 1987, 243 (2), 492-500.
16. Cowan, A.; Zhu, X. Z.; Mosberg, H. I.; Omnaas, J. R.; Porreca, F., Direct dependence studies in rats with agents selective for different types of opioid receptor.

J Pharmacol Exp Ther 1988, 246 (3), 950-5.
17. Szeto, H. H.; Soong, Y.; Wu, D.; Olariu, N.; Kett, A.; Kim, H.; Clapp, J. F., Respiratory depression after intravenous administration of delta-selective opioid peptide analogs. Peptides 1999, 20 (1), 101-5.
18. Henry, M. S.; Gendron, L.; Tremblay, M. E.; Drolet, G., Enkephalins: Endogenous Analgesics with an Emerging Role in Stress Resilience. Neural Plast 2017, 2017, 1546125.
19. Corder, G.; Castro, D. C.; Bruchas, M. R.; Scherrer, G., Endogenous and Exogenous Opioids in Pain. Annu Rev Neurosci 2018, 41, 453-473.
20. Roques, B. P.; Fournie-Zaluski, M. C.; Wurm, M., Inhibiting the breakdown of endogenous opioids and cannabinoids to alleviate pain. Nat Rev Drug Discov 2012, 11 (4), 292-310.
21. Stein, C.; Schafer, M.; Machelska, H., Attacking pain at its source: new perspectives on opioids. Nat Med 2003, 9 (8), 1003-8.
22. Lindqvist, A.; Jonsson, S.; Hammarlund-Udenaes, M., Exploring Factors Causing Low Brain Penetration of the Opioid Peptide DAMGO through Experimental Methods and Modeling. Mol Pharm 2016, 13 (4), 1258-66.
23. Deekonda, S.; Cole, J.; Sunna, S.; Rankin, D.; Largent-Milnes, T. M.; Davis, P.; BassiriRad, N. M.; Lai, J.; Vanderah, T. W.; Porecca, F.; Hruby, V. J., Enkephalin analogues with N-phenyl-N-(piperidin-2-ylmethyl)propionamide derivatives: Synthesis and biological evaluations. Bioorg Med Chem Lett 2016, 26 (1), 222-7.
24. Cros, C. D.; Toth, I.; Blanchfield, J. T., Lipophilic derivatives of leu-enkephalinamide: in vitro permeability, stability and in vivo nasal delivery. Bioorg Med Chem 2011, 19 (4), 1528-34.
25. Geiger, R.; Bickel, M.; Teetz, V.; Alpermann, H. G., Central and peripheral action of enkephalin analogues. Hoppe Seylers Z Physiol Chem 1983, 364 (11), 1555-62.
26. Bilsky, E. J.; Egleton, R. D.; Mitchell, S. A.; Palian, M. M.; Davis, P.; Huber, J. D.; Jones, H.; Yamamura, H. I.; Janders, J.; Davis, T. P.; Porreca, F.; Hruby, V. J.; Polt, R., Enkephalin glycopeptide analogues produce analgesia with reduced dependence liability. J Med Chem 2000, 43 (13), 2586-90.
27. Deekonda, S.; Wugalter, L.; Rankin, D.; Largent-Milnes, T. M.; Davis, P.; Wang, Y.; Bassirirad, N. M.; Lai, J.; Kulkarni, V.; Vanderah, T. W.; Porreca, F.; Hruby, V. J., Design and synthesis of novel bivalent ligands (MOR and DOR) by conjugation of enkephalin analogues with 4-anilidopiperidine derivatives. Bioorg Med Chem Lett 2015, 25 (20), 4683-8.
28. Masand, G.; Hanif, K.; Sen, S.; Ahsan, A.; Maiti, S.; Pasha, S., Synthesis, conformational and pharmacological studies of glycosylated chimeric peptides of Met-enkephalin and FMRFa. Brain Res Bull 2006, 68 (5), 329-34.
29. Zadina, J. E.; Kastin, A. J.; Hackler, L.; Chang, S. L., Cyclic analogues of Tyr-W-MIF-1 with prolonged analgesic activity and potency comparable to DAMGO and morphine. Peptides 1994, 15 (8), 1567-9.
30. Mollica, A.; Costante, R.; Novellino, E.; Stefanucci, A.; Pieretti, S.; Zador, F.; Samavati, R.; Borsodi, A.; Benyhe, S.; Vetter, I.; Lewis, R. J., Design, Synthesis and Biological Evaluation of Two Opioid Agonist and Cav 2.2 Blocker Multitarget Ligands. Chem Biol Drug Des 2015, 86 (2), 156-62.
31. Christie, M. P.; Simerska, P.; Jen, F. E.; Hussein, W. M.; Rawi, M. F.; Hartley-Tassell, L. E.; Day, C. J.; Jennings, M. P.; Toth, I., A drug delivery strategy: binding enkephalin to asialoglycoprotein receptor by enzymatic galactosylation. PLoS One 2014, 9 (4), e95024.
32. Zhou, L.; Zhang, Q.; Stein, C.; Schafer, M., Contribution of opioid receptors on primary afferent versus sympathetic neurons to peripheral opioid analgesia. J Pharmacol Exp Ther 1998, 286 (2), 1000-6.
33. Ji, R. R.; Zhang, Q.; Law, P. Y.; Low, H. H.; Elde, R.; Hokfelt, T., Expression of mu-, delta-, and kappa-opioid receptor-like immunoreactivities in rat dorsal root ganglia after carrageenan-induced inflammation. J Neurosci 1995, 15 (12), 8156-66.
34. Rittner, H. L.; Brack, A.; Machelska, H.; Mousa, S. A.; Bauer, M.; Schafer, M.; Stein, C., Opioid peptide-expressing leukocytes: identification, recruitment, and simultaneously increasing inhibition of inflammatory pain. Anesthesiology 2001, 95 (2), 500-8.
35. Roques, B. P., Novel approaches to targeting neuropeptide systems. Trends Pharmacol Sci 2000, 21 (12), 475-83.
36. Hokfelt, T.; Bartfai, T.; Bloom, F., Neuropeptides: opportunities for drug discovery. Lancet Neurol 2003, 2 (8), 463-72.
37. Popik, P.; Kamysz, E.; Kreczko, J.; Wrobel, M., Human opiorphin: the lack of physiological dependence, tolerance to antinociceptive effects and abuse liability in laboratory mice. Behav Brain Res 2010, 213 (1), 88-93.
38. Raffa, R. B.; Pergolizzi, J. V., Jr.; Taylor, R., Jr.; Ossipov, M. H.; By the, N. R. G., Indirect-acting strategy of opioid action instead of direct receptor activation: dual-acting enkephalinase inhibitors (DENKIs). J Clin Pharm Ther 2018, 43 (4), 443-449.
39. Fournie-Zaluski, M. C.; Chaillet, P.; Bouboutou, R.; Coulaud, A.; Cherot, P.; Waksman, G.; Costentin, J.; Roques, B. P., Analgesic effects of kelatorphan, a new highly potent inhibitor of multiple enkephalin degrading enzymes. Eur J Pharmacol 1984, 102 (3-4), 525-8.
40. Roques, B. P.; Noble, F.; Dauge, V.; Fournie-Zaluski, M. C.; Beaumont, A., Neutral endopeptidase 24.11: structure, inhibition, and experimental and clinical pharmacology. Pharmacol Rev 1993, 45 (1), 87-146.
41. Noble, F.; Soleilhac, J. M.; Soroca-Lucas, E.; Turcaud, S.; Fournie-Zaluski, M. C.; Roques, B. P., Inhibition of the enkephalin-metabolizing enzymes by the first systemically active mixed inhibitor prodrug RB 101 induces potent analgesic responses in mice and rats. J Pharmacol Exp Ther 1992, 261 (1), 181-90.
42. Thanawala, V.; Kadam, V. J.; Ghosh, R., Enkephalinase inhibitors: potential agents for the management of pain. Curr Drug Targets 2008, 9 (10), 887-94.
43. Bonnard, E.; Poras, H.; Fournie-Zaluski, M. C.; Roques, B. P., Preventive and alleviative effects of the dual enkephalinase inhibitor (Denki) PL265 in a murine model of neuropathic pain. Eur J Pharmacol 2016, 788, 176-182.
44. Machelska, H.; Stein, C., Immune mechanisms in pain control. Anesth Analg 2002, 95 (4), 1002-8, table of contents.
45. Stein, C.; Hassan, A. H.; Przewlocki, R.; Gramsch, C.; Peter, K.; Herz, A., Opioids from immunocytes interact with receptors on sensory nerves to inhibit nociception in inflammation. Proc Natl Acad Sci USA 1990, 87 (15), 5935-9.
46. Poonyachoti, S.; Kulkarni-Narla, A.; Brown, D. R., Chemical coding of neurons expressing delta- and kappa-opioid receptor and type I vanilloid receptor immunoreactivities in the porcine ileum. Cell Tissue Res 2002, 307 (1), 23-33.
47. Le Bars, D.; Gozariu, M.; Cadden, S. W. Animal Models of Nociception.

Pharmacological Reviews. 2001, pp 597-652.
48. Tjslsen, A.; Berge, O. G.; Hunskaar, S.; Rosland, J. H.; Hole, K. The Formalin Test: An Evaluation of the Method. Pain. 1992, pp 5-17.
49. Dahan, A.; Van Der Schrier, R.; Smith, T.; Aarts, L.; Van Velzen, M.; Niesters, M. Averting Opioid-Induced Respiratory Depression without Affecting Analgesia. Anesthesiology. 2018, pp 1027-1037.
50. Hill, R.; Lyndon, A.; Withey, S.; Roberts, J.; Kershaw, Y.; Maclachlan, J.; Lingford-Hughes, A.; Kelly, E.; Bailey, C.; Hickman, M.; et al. Ethanol Reversal of Tolerance to the Respiratory Depressant Effects of Morphine. Neuropsychopharmacology 2016, 41 (3), 762-773.
51. Hill, R.; Santhakumar, R.; Dewey, W.; Kelly, E.; Henderson, G. Fentanyl Depression of Respiration: Comparison with Heroin and Morphine. Br. J. Pharmacol. 2020, 177 (2), 254-266.
52. Yamazaki, M.; Suzuki, T.; Narita, M.; Lipkowski, A. W. The Opioid Peptide Analogue Biphalin Induces Less Physical Dependence than Morphine. Life Sci. 2001, 69 (9), 1023-1028.
53. Mello, N. K.; Mendelson, J. H. Self-Administration of an Enkephalin Analog by Rhesus Monkey. Pharmacol. Biochem. Behav. 1978.
54. Lowery, J. J.; Raymond, T. J.; Giuvelis, D.; Bidlack, J. M.; Polt, R.; Bilsky, E. J. In Vivo Characterization of MMP-2200, a Mixed δ/μ Opioid Agonist, in Mice. J. Pharmacol. Exp. Ther. 2011, 336 (3), 767-778.
55. Fitzgerald P J, Yen J Y, Watson B O. Stress-sensitive antidepressant-like effects of ketamine in the mouse forced swim test. PLoS One [Internet]. 2019 Apr. 1 [cited 2021 Oct. 29]; 14(4):e0215554.
56. Can A, Dao D T, Arad M, Terrillion C E, Piantadosi S C, Gould T D. The Mouse Forced Swim Test. JoVE (Journal Vis Exp [Internet]. 2012 Jan. 29 [cited 2021 Nov. 8]; (59):e3638.
57. Can A, Dao D T, Terrillion C E, Piantadosi S C, Bhat S, Gould T D. The Tail Suspension Test. JoVE (Journal Vis Exp [Internet]. 2012 Jan. 28 [cited 2021 Nov. 8]; (59):e3769.
58. Iyer K A, Alix K, Eltit J M, Solis E, Pan X, Argade M D, et al. Multi-modal antidepressant-like action of 6- and 7-chloro-2-aminodihydroquinazolines in the mouse tail suspension test. Psychopharmacology (Berl) [Internet]. 2019 Jul. 1 [cited 2021 Nov. 8]; 236(7):2093-104.

1. A compound of Formula I: 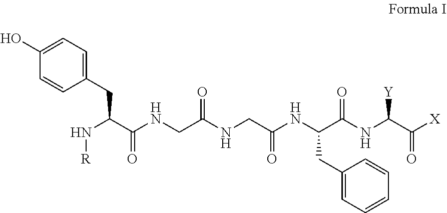
wherein R is independently optionally substituted C₁₋₈ alkyl, C₁₋₈ alkenyl, acyl or phenyl;
Y is CH₂CH(CH₃)₂ or CH₂CH₂SCH₃; and
X is independently OH, NH₂, NH—NH₂, NH—NHOH, halo, thio, NCH₃, N(CH₃)₂, triazole, tetrazole, alkoxy or a methyl ester, or a pharmaceutically acceptable salt thereof.

2. The compound of claim 1 wherein R is optionally substituted with halo or thio.

3. The compound of claim 1 wherein R is acyl or C₁₋₈ alkyl.

4. The compound of claim 1 wherein R is alkanoyl or benzoyl.

5. The compound of claim 1 wherein R is pivaloyl or t-butyl.

6. The compound of claim 1 wherein R is 2-(tert-butyl)benzoyl, 2,4,6-tri-tert-butylbenzoyl, 2,2-diphenylpropanal, 2-hydroxy-2,2-diphenylacetyl, 3,3-dimethyl-2-phenylbutanoyl, 2-methyl-2-phenylpropanoyl, 2-hydroxy-2-phenylpropanoyl, 2-methyl-2-phenylpropanoyl, 2-amino-2,2-diphenylacetyl, dihydroxyl phenyl acetyl, trihydroxyl phenyl acetyl, dimethoxy phenylacetyl or trimethoxy phenylacetyl.

7. The compound of claim 1 wherein R is (CF₃)₂—CHOH—CO, HO—CHCH₂OH—CO, (CH₃)₂—COH—CO, NH₂—CH—(CH₃)₂—CO, (CH₃)₃—CH₂CO, or (CH₃)₂—CHOH—CH₂—.

8. The compound of claim 1 wherein R is

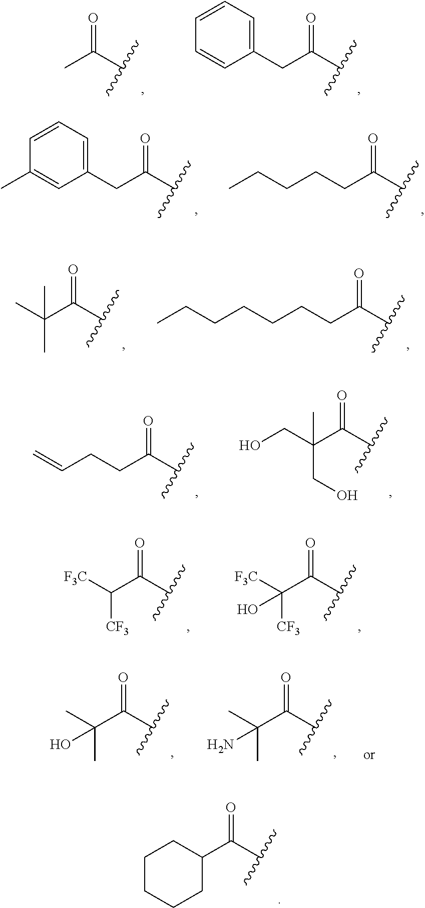

9. The compound of claim 1 wherein R is

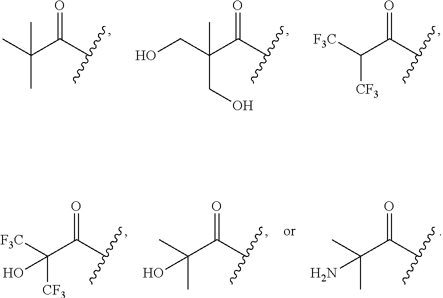

10. The compound of claim 1 wherein R is

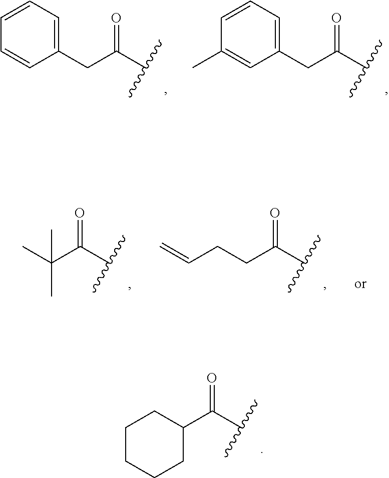

11. The compound of claim 1 wherein R is

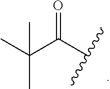 [SMILES: *C(=O)C(C)(C)C]

12. The compound of claim 1 wherein X is independently OH or methyl ester.

13. The compound of claim 1 wherein
R is 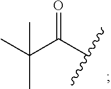 [SMILES: *C(=O)C(C)(C)C]
Y is CH₂CH(CH₃)₂; and
X is independently OH or a methyl ester, or a pharmaceutically acceptable salt thereof.

14. The compound of claim 1 wherein the compound has the following structure:

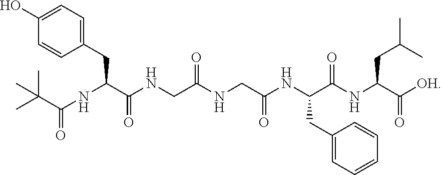 [SMILES: CC(C)C[C@H](NC(=O)[C@H](Cc1ccccc1)NC(=O)CNC(=O)CNC(=O)[C@H](Cc1ccc(O)cc1)NC(=O)C(C)(C)C)C(=O)O]

15. A pharmaceutical composition comprising the compound of claim 1.

16. A method of treating pain, anxiety, a mood disorder or depression, comprising administering the compound of claim 1 to a subject in need thereof.

17. A method of treating pain, anxiety, a mood disorder or depression, comprising administering the pharmaceutical composition of claim 15 to a subject in need thereof.

18. A commercial package comprising the compound of claim 1, and instructions for treating pain, anxiety, a mood disorder or depression.

19. The method of claim 16 wherein the pain is acute pain, chronic pain, inflammatory pain, or neuropathic pain.

20. The method of claim 16 wherein the subject is a human.
